(19) United States
(12) Patent Application Publication
(10) Pub. No.: US 2011/0275918 A1
(43) Pub. Date: Nov. 10, 2011

(54) BODY FLUID COLLECTING DEVICE FOR EFFICIENTLY COLLECTING BODY FLUID AND BODY FLUID ANALYZER FOR ACCURATE ANALYSIS

(75) Inventors: Hideyuki YAMASHITA, Kyoto-shi (JP); Tomohiko MATSUSHITA, Kyoto-shi (JP); Midori TANIGUCHI, Ahuachapan (SV); Satoshi NAKAJIMA, Kyoto-shi (JP); Muneo TOKITA, Nagaokakyo-shi (JP)

(73) Assignee: OMRON HEALTHCARE CO., LTD., KYOTO-SHI (JP)

(21) Appl. No.: 13/183,172

(22) Filed: Jul. 14, 2011

Related U.S. Application Data
(63) Continuation of application No. PCT/JP2010/050717, filed on Jan. 21, 2010.

(30) Foreign Application Priority Data
Jan. 23, 2009 (JP) 2009-013126

(51) Int. Cl. A61M 37/00; A61B 5/00; A61M 5/168; A61M 5/00; A61M 5/142

(52) U.S. Cl. 600/345; 604/93.01; 604/151; 604/244; 604/246; 600/573; 604/20; 604/22; 600/309

(57) ABSTRACT

In a body fluid analyzer, a body fluid extracting unit for holding medicine to promote discharge of body fluid and collecting body fluid is arranged at a lower surface of a body fluid collecting chip. A body fluid discharge promoting medicine storage unit is connected to a medicine injecting hole formed in the body fluid collecting chip. A test unit is arranged in the body fluid collecting chip, and the body fluid extracting unit and the test unit are connected by a body fluid feeding path. A discarding body fluid storage unit is connected to the exit of the body fluid feeding path. A medicine supplying mechanism including a pump is operatively connected.

### TECHNICAL FIELD

The present invention relates to a body fluid collecting device and a body fluid analyzer. Specifically, the present invention relates to a body fluid collecting device and a body fluid collecting method for promoting body fluid extraction, and collecting or obtaining body fluid (sweat, cell fluid, tissue fluid of living tissue, etc.). The present invention also relates to a body fluid analyzer for specifying type and concentration of the component in the collected body fluid.

### BACKGROUND ART

Disorders occurs in the immune mechanism, which a human originally has, thus causing various diseases due to change in dietary habit of a human in the recent years, lack of exercise, physical and mental burden due to extreme fatigue and stress, smoking, drinking, and the like. Since lifestyle habits are deeply related to the occurrence and progress of the diseases related thereto, such diseases are generally called lifestyle related diseases. The lifestyle related diseases include obesity, dyslipidemia, diabetes, and high blood pressure, and also cancer, apoplexy, liver ailment, and osteoporosis.

In particular, the number of patients with diabetes is significantly increasing worldwide. In Japan, the number of patients with diabetes is said to be 6.9 million in 2005 and 170 million in the world. Diabetes usually do not have certain symptoms, and thus not many people are being treated even after being diagnosed as diabetes. If not treated, the disease slowly progresses in the body, and leads to many complications that may even result in loss of sight or amputation of the lower limb. Therefore, to what extent the disease is progressing needs to be checked (tested) periodically. The sign of complication needs to be found at an early stage while continuing the check and confirming good and bad of the blood sugar control.

The diabetes is a disease in which the amount of insulin secreted in the pancreas lacks, so that the sugar is not used and overflows into the blood. Therefore, the blood sugar needs to be periodically monitored, and an appropriate amount of insulin needs to be injected into the body in view of the result.

Currently, the blood is actually collected, and the enzyme reaction is detected electrochemically or with color to monitor the sugar. However, there are a number of concerning matters to taking blood. One is the physical and mental burden of invading the skin and collecting blood. The patient with diabetes is required to carry out measurement several times in one day such as after meal. In other words, the blood needs to be collected by inserting needle into the skin several times a day. The blood has a possibility of infection. In severe patients, the monitoring needs to be carried out even during sleep, and continuous monitoring is strongly desired. Therefore, a sensor capable of monitoring the blood sugar level without inserting needle into the skin (i.e., non-invasively) is strongly desired.

(Invention of Patent Document 1)

Japanese Unexamined Patent Publication No. 9-5296 (patent document 1) discloses an analyzing sensor for measuring the blood sugar level. As shown in FIG. 1A, the analyzing sensor has a bio sensor chip 12 attached in a recess formed on the inner surface of a holding member 11. As shown in FIG. 1B, the bio sensor chip 12 has a pair of comb tooth-like electrodes 14a, 14b formed at the lower surface of the substrate 13, and the surface covered with a protective electrode 15, where an enzyme film 16 and a separation film 17 are stacked on the lower surface.

The analyzing sensor is used by pressure contacting a surface on the side the biosensor chip 12 is arranged to the surface of the skin 18. The analyzing sensor pressure contacted to the skin 18 obtains sweat 19 secreted from the skin surface through the separation film 17, reacts the enzyme in the enzyme film 16 and the component contained in the sweat 19, and detects the electric signal generated in the reaction with the comb tooth-like electrodes 14a, 14b. The type and amount of component of the sweat are specified based on the detection signal. The blood sugar level can be calculated by the analyzing sensor by measuring the glucose amount in the sweat in a non-invasive manner.

However, in the analyzing sensor, the amount of perspiration from the skin is small since the sweat is secreted from the skin by natural perspiration, and hence a long time is required to collect the amount of sweat necessary for the test.

(Inventions of Non-Patent Document 1, Patent Documents 2, 3)

Therefore, an iontophoresis method of promoting perspiration using medicine (body fluid discharge promoting medicine) is proposed. The sweat collecting system disclosed in the catalogue (non-patent document 1) of “Sweat Collection System” from Macroduct Co. uses the iontophoresis method, and includes an administration electrode on a cathode side, an administration electrode on an anode side, and a sweat collector. The overall configuration of the sweat collecting system is disclosed in non-patent document 1, the administration electrode is disclosed in U.S. Pat. No. 4,383,529 (patent document 2), and the sweat collector is disclosed in U.S. Pat. No. 4,542,751 (patent document 3).

When carrying out tests with the sweat collecting system, the body fluid discharge promoting medicine is attached to the administration electrode on the cathode side, the administration electrode on the cathode side and the administration electrode on the anode side are respectively attached to the arm with the belt, and thereafter, voltage is applied between the administration electrodes to flow current to the body fluid discharge promoting medicine thus promoting the perspiration from the arm. The administration electrodes are then detached from the arm, and the sweat collector having a wrist watch shape is attached to the area where the body fluid discharge promoting medicine was attached. The sweat extracted from the arm at the lower surface of the sweat collector is taken up with a spiral shaped collecting tube arranged in the sweat collector. When sufficient sweat is collected, the sweat in the collecting tube is taken up by the dispenser, and such sweat is injected to the test device to carry out tests with the test device.

According to such sweat collecting system, the amount of perspiration can be increased by using the body fluid discharge promoting medicine, and hence the sweat can be collected in a short period of time. However, although the time for extracting the necessary amount of sweat from the arm reduces, the tasks of reattaching the administration electrode and the sweat collector, transferring the collected sweat from the collecting tube to the dispenser, and further injecting to the test device become troublesome, and overall time required for the test becomes longer.

Furthermore, the test cannot be carried out continuously for a plurality of times since reattachment of the administration electrode and the sweat collector to the arm is required every time the test is carried out in the sweat collecting system.

(Patent Document 4)

In the analyzer disclosed in Japanese Unexamined Patent Publication No. 2005-246054 (patent document 4), normal saline solution is brought into contact with the skin, the body fluid (tissue fluid) is collected in the normal saline solution with the reverse iontophoresis method, and a specific component (glucose etc.) in the body fluid is measured.

In the analyzer of patent document 4, the extracted body fluid is collected in the normal saline solution, so that the body fluid and the normal saline solution coexist and the influence on the measurement accuracy is large. Therefore, if a specific component in the body fluid is low concentration, the measurement thereof is difficult. The component in the normal saline solution may become a noise and lower the measurement accuracy depending on the type of specific component to be measured. Moreover, in the analyzer, the measurement cannot be repeatedly carried out while being attached to the body since a structure of discarding the normal saline solution mixed with body fluid is not adopted.

Patent Document 1: Japanese Unexamined Patent Publication No. 9-5296

Patent Document 2: U.S. Pat. No. 4,383,529

Patent Document 3: U.S. Pat. No. 4,542,751

Patent Document 4: Japanese Unexamined Patent Publication No. 2005-246054

Catalogue (3700-Macroduct Sweat Collection System Brochure) of Sweat Collection System from Macroduct Co. [online] searched Dec. 20, 2008, [Internet] “http://www.discovery-diagnostics.com/pdf/Macroduct-Sweat-Chek.pdf”

### DISCLOSURE OF THE INVENTION

### Problems to be Solved by the Invention

One object of the present invention is to provide a body fluid collecting device and a body fluid collecting method capable of collecting body fluid with less mixture in which the body fluid is less likely to mix into the body fluid discharge promoting medicine while collecting body fluid efficiently and non-invasively using the body fluid discharge promoting medicine. Another object is to provide a body fluid analyzer capable of accurately carrying out tests by using body fluid with less foreign substances. Another further object is to provide a body fluid analyzer capable of reducing the time required for the tests and repeatedly and continuously carrying out tests.

### Means for Solving the Problem

In order to solve the above problem, in accordance with one aspect of the present invention, a body fluid collecting device relates to a body fluid collecting device for extracting and collecting body fluid from under the skin or from the body of a subject, the body fluid collecting device including a body fluid extracting unit having a function of holding a body fluid discharge promoting medicine for discharging the body fluid from under the skin or from the body on a body fluid discharge site and a function of collecting the body fluid extracted from a site where the body fluid discharge promoting medicine is administered; a medicine collecting passage having one end opened at the body fluid extracting unit, for collecting or discarding the body fluid discharge promoting medicine; and a medicine collecting mechanism for collecting or discarding the body fluid discharge promoting medicine remaining in the body fluid extracting unit after administered to under the skin or into the body through the medicine collecting passage.

The body fluid collecting device can non-invasively collect the body fluid without giving pain to the subject according to such configuration. Furthermore, the discharge of the body fluid can be promoted using the body fluid discharge promoting medicine. The collecting efficiency of the body fluid thus can be enhanced, and the necessary amount of body fluid can be collected in a short time. Furthermore, the body fluid collecting device includes the medicine collecting passage and the medicine collecting mechanism for collecting or discarding the body fluid discharge promoting medicine in the body fluid extracting unit, so that after supplying the body fluid discharge promoting medicine to the body fluid extracting unit and before collecting the body fluid, the body fluid discharge promoting medicine remaining in the body fluid extracting unit can be discharged from the body fluid extracting unit. Thus, the body fluid discharge promoting medicine is less likely to mix with the body fluid and make the body fluid thin, the measurement can be carried out with only the body fluid, and the test accuracy of the specific component in the body fluid can be enhanced. A risk that the component of the body fluid discharge promoting medicine may become a noise and lower the test accuracy is reduced. In particular, the specific component can be measured even if the specific component in the body fluid is low concentration.

The body fluid collecting device further preferably includes a medicine injection passage for injecting the body fluid discharge promoting medicine to the body fluid extracting unit. According to such body fluid collecting device, the body fluid discharge promoting medicine does not need to be applied to the body fluid extracting unit in advance, and the body fluid discharge promoting medicine can be injected from the medicine injection passage to the body fluid extracting unit after attaching the body fluid collecting device to the arm or the like of the user. According to such body fluid collecting device, the body fluid can be repeatedly collected over plural times with the body fluid collecting device attached to the arm or the like.

The body fluid discharge promoting medicine may be automatically injected using the medicine supplying mechanism such as a pump, or the body fluid discharge promoting medicine may be injected by hand using a syringe or an injector to the medicine injection passage.

The body fluid collecting device more preferably includes a body fluid discharge promoting medicine storage unit for storing the body fluid discharge promoting medicine; and a medicine supplying mechanism for supplying the body fluid discharge promoting medicine stored in the body fluid discharge promoting medicine storage unit from the medicine injection passage to the body fluid extracting unit. According to such body fluid collecting device, the body fluid discharge promoting medicine in the body fluid discharge promoting medicine storage unit can be automatically supplied to the body fluid extracting unit by the medicine supplying mechanism, and the body fluid discharge promoting medicine can be further automatically discharged by the medicine collecting mechanism, and hence the test can be automated and the test time can be reduced.

If the body fluid discharge promoting medicine is liquid, the pump may be used for the medicine supplying mechanism.

More preferably, in the body fluid collecting device, the body fluid discharge promoting medicine is liquid; the body fluid discharge promoting medicine storage unit is a breakable liquid container in which the body fluid discharge promoting medicine is enclosed; and the medicine supplying mechanism is a breaking tool for breaking the liquid container. According to such body fluid collecting device, a constant amount of body fluid discharge promoting medicine can be supplied with a simple structure.

The body fluid collecting device preferably includes a valve for opening and closing the passage is arranged on the medicine injection passage. According to such body fluid collecting device, the body fluid discharge promoting medicine can be prevented from being drawn out from the body fluid discharge promoting medicine storage unit by closing the valve when discharging the body fluid discharge promoting medicine in the body fluid extracting unit or when discharging the body fluid in the test unit.

In the body fluid collecting device, the body fluid discharge promoting medicine is preferably liquid; and the medicine collecting mechanism collects or discards the body fluid discharge promoting medicine from the body fluid extracting unit by a method selected from a method of pushing out the body fluid discharge promoting medicine by air sent to the body fluid extracting unit using a pump, a method of pushing out the body fluid discharge promoting medicine by the body fluid discharged from under the skin or from the body to the body fluid extracting unit, and a method of aspirating the body fluid discharge promoting medicine using a pump. According to such body fluid collecting device, the body fluid discharge promoting medicine can be collected through various methods.

In the body fluid collecting device, the body fluid discharge promoting medicine is preferably liquid; and the medicine collecting mechanism feeds a volatile liquid to the body fluid extracting unit to substitute or mix with the body fluid discharge promoting medicine, and volatilizes the volatile liquid. According to such body fluid collecting device, the body fluid discharge promoting medicine can be more easily removed since the body fluid discharge promoting medicine can be removed from the body fluid extracting unit by volatilizing the volatile liquid or the volatile liquid mixed with the body fluid discharge promoting medicine.

The body fluid collecting device further preferably includes a body fluid collecting passage, having one end opened at the body fluid extracting unit, for collecting the body fluid; and a body fluid collecting mechanism for collecting the body fluid collected in the body fluid extracting unit through the body fluid collecting passage. According to such body fluid collecting device, the body fluid collected at the body fluid extracting unit by the body fluid collecting mechanism can be automatically collected, and the body fluid discharge promoting medicine can be automatically discharged from the body fluid extracting unit by the medicine collecting mechanism, so that the test can be automated and the test time can be reduced.

The body fluid collecting device preferably has an inner wall surface of the body fluid extracting unit inclined so that an open end of the body fluid collecting passage is positioned at top of the body fluid extracting unit at a cross-section of the body fluid extracting unit including a direction perpendicular to the body fluid discharge site. According to such body fluid collecting device, the body fluid collecting passage is less likely to get blocked by the body fluid discharge site (skin) when attached to the body fluid discharge site of the subject. Furthermore, the consumed amount of body fluid discharge promoting medicine can be reduced and the reduction of the measurement time can be contributed since the volume of the body fluid extracting unit is reduced.

The body fluid collecting device preferably includes a valve for opening and closing the passage on the body fluid collecting passage. According to such body fluid collecting device, the discarding body fluid in the discarding body fluid storage unit can be prevented from being drawn out and back flowing by closing the valve when discharging the body fluid discharge promoting medicine in the body fluid extracting unit.

The body fluid collecting device preferably includes a mechanism for generating ultrasonic vibration between the body fluid discharge promoting medicine held at the body fluid extracting unit and the body fluid discharge site. According to such body fluid collecting device, the penetration of the body fluid discharge promoting medicine to the body fluid discharge site can be promoted by generating the ultrasonic vibration, so that the extraction speed of the body fluid can be increased and more body fluid can be collected in a shorter time compared to when simply using the body fluid discharge promoting medicine.

The body fluid collecting device preferably includes at least two administration electrodes for flowing current between the body fluid discharge promoting medicine held in the body fluid extracting unit and the body fluid discharge site. According to such body fluid collecting device, the penetration of the body fluid discharge promoting medicine can be promoted by flowing current between the body fluid discharge promoting medicine and the body fluid discharge site, so that the extraction speed of the body fluid can be increased and more body fluid can be collected in a shorter time compared to when simply using the body fluid discharge promoting medicine. Furthermore, even the body fluid discharge promoting medicine that is hard to be penetrated to under the skin or into the body can be easily penetrated to under the skin or into the body by applying voltage.

The body fluid collecting device preferably has at least a portion close to the body fluid extracting unit of the medicine injection passage for injecting the body fluid discharge promoting medicine to the body fluid extracting unit formed by a conductive material so that the medicine injection passage also serves as either one of the administration electrodes. According to such body fluid collecting device, the structure of the body fluid collecting device can be simplified since the medicine injection passage also serves as the administration electrode.

The body fluid collecting device preferably has at least one of the administration electrodes arranged to contact the body fluid discharge promoting medicine held in the body fluid extracting unit and at a position different from the medicine injection passage.

The body fluid collecting device preferably has a conductive film arranged on the surface body fluid extracting unit, and at least one of the administration electrodes is formed by the conductive film. According to the body fluid collecting device, the contacting area of the administration electrode and the body fluid discharge promoting medicine can be widened, so that the disconnection state by the air bubbles in the body fluid discharge promoting medicine can be prevented and a state in which the voltage is not applied on the body fluid discharge promoting medicine can be prevented.

In the body fluid collecting device, the body fluid discharge promoting medicine is preferably medicine containing pilocarpine or acetylcholine. According to such body fluid collecting device, the perspiration from the body fluid discharge site can be promoted and sweat can be collected in a short time.

In accordance with another aspect of the present invention, a body fluid collecting method is a body fluid collecting method for collecting body fluid using the body fluid collecting device, the body fluid collecting method including the steps of attaching the body fluid collecting device to a body fluid discharge site; and sequentially executing with the body fluid collecting device processes of administering a body fluid discharge promoting medicine held in a body fluid extracting unit to under the skin or into the body; removing the body fluid discharge promoting medicine remaining in the body fluid extracting unit with a medicine collecting mechanism; and collecting the body fluid extracted from the site where the body fluid discharge promoting medicine is administered with the body fluid extracting unit.

The body fluid collecting method can non-invasively collect the body fluid without giving pain to the subject. Furthermore, the collecting efficiency of the body fluid can be enhanced and the necessary amount of body fluid can be collected in a short time since the discharging of the body fluid is promoted using the body fluid discharge promoting medicine. Furthermore, the body fluid collecting device includes the medicine collecting passage and the medicine collecting mechanism for collecting or discarding the body fluid discharge promoting medicine in the body fluid extracting unit, so that after supplying the body fluid discharge promoting medicine to the body fluid extracting unit and before collecting the body fluid, the body fluid discharge promoting medicine remaining in the body fluid extracting unit can be discharged from the body fluid extracting unit. Thus, the body fluid discharge promoting medicine is less likely to mix with the body fluid and make the body fluid thin, the measurement can be carried out with only the body fluid, and the test accuracy of the specific component in the body fluid can be enhanced. It is less possible that the component of the body fluid discharge promoting medicine becomes a noise and lowers the test accuracy. In particular, the specific component can be measured even if the specific component in the body fluid is low concentration.

In accordance with another aspect of the present invention, a body fluid analyzer is a body fluid analyzer for detecting or measuring a specific component in a body fluid collected from under the skin or from the body of a subject, wherein the body fluid collecting device is arranged as a body fluid collecting unit for collecting from under the skin or from the body of the subject; and the body fluid analyzer further includes a test unit for detecting or measuring the specific component in the body fluid collected by the body fluid extracting unit arranged in the body fluid collecting unit.

The body fluid analyzer can continuously test the specific component in the body fluid with the same device without reattaching the body fluid analyzer since the body fluid analyzer includes the body fluid collecting unit and the test unit. The body fluid analyzer can non-invasively test the specific component in the body fluid without giving pain to the subject. Furthermore, the collecting efficiency of the body fluid can be enhanced, the necessary amount of body fluid can be collected in a short time, and the time required for the test can be reduced since the discharging of the body fluid is promoted using the body fluid discharge promoting medicine. Furthermore, the body fluid collecting device includes the medicine collecting passage and the medicine collecting mechanism for collecting or discarding the body fluid discharge promoting medicine in the body fluid extracting unit, so that after supplying the body fluid discharge promoting medicine to the body fluid extracting unit and before collecting the body fluid, the body fluid discharge promoting medicine remaining in the body fluid extracting unit can be discharged from the body fluid extracting unit. Thus, the body fluid discharge promoting medicine is less likely to mix with the body fluid and make the body fluid thin, the measurement can be carried out with only the body fluid, and the test accuracy of the specific component in the body fluid can be enhanced. The component of the body fluid discharge promoting medicine is less likely to become a noise and lower the test accuracy, so that the sensitivity of the body fluid analyzer can be enhanced. In particular, the specific component can be measured even if the specific component in the body fluid is low concentration.

In the body fluid analyzer, the test unit preferably includes an enzyme that specifically reacts with the specific component in the body fluid, and a test electrode; and a signal based on a current generated from a reaction between the specific component in the body fluid and the enzyme is detected by the test electrode to detect or measure the specific component in the body fluid. According to such body fluid analyzer, the current flowing between the test electrodes changes by the oxidation-reduction reaction of the specific component and the enzyme, so that the presence or absence, and the amount (concentration) of the specific component can be measured based on such current value.

In the body fluid analyzer, alternatively, the test unit preferably includes an enzyme and a color forming dye that specifically react with the specific component in the body fluid; and a color reaction between the specific component in the body fluid and the enzyme and the color forming dye is optically detected to detect or measure the specific component in the body fluid. According to such body fluid analyzer, the color reaction between the specific component in the body fluid and the enzyme and the color forming dye is optically detected, and the presence or absence, and the amount (concentration) of the specific component can be measured based on wavelength spectrum and the like.

In the body fluid analyzer, two or more test units are more preferably arranged; and the test units include enzymes of different type from each other. According to such body fluid analyzer, different specific components can be tested with each enzyme, a plurality of specific components can be simultaneously (in one time) measured, and the test can be efficiently carried out.

In the body fluid analyzer, two or more test units are more preferably arranged; and the test units include same type of enzyme. According to such body fluid analyzer, the same specific components can be tested with each enzyme, so that one specific component can be measured over a plurality of times at one time, and higher measurement accuracy can be achieved.

In the body fluid analyzer, four or more test units are more preferably arranged; and with a combination of test units having different types of enzymes from each other as one set, the one set of test units is arranged in plurals. According to such body fluid analyzer, a plurality of specific components can be measured in one time, the same specific component can be measured over a plurality of times, and higher efficiency of the measurement task and higher measurement accuracy can be achieved.

A calibration fluid storage unit for storing calibration fluid for calibrating the test unit; and a calibration fluid supplying mechanism for supplying the calibration fluid stored in the calibration fluid storage unit to the test unit are preferably arranged. According to such body fluid analyzer, the measurement value of the test unit can be calibrated using the calibration fluid, and higher measurement accuracy of the body fluid analyzer can be achieved. Furthermore, the calibration task can be automated since the calibration fluid in the calibration fluid storage unit can be fed to the test unit by the calibration fluid supplying mechanism.

The body fluid analyzer further preferably includes a body fluid collecting passage, having one end opened at the body fluid extracting unit, for collecting the body fluid; a discarding body fluid storage unit for discarding the body fluid after the test; and a body fluid collecting mechanism for collecting the body fluid collected at the body fluid extracting unit through the body fluid collecting passage, and discarding the body fluid after the test by the test unit to the discarding body fluid storage unit. According to such body fluid analyzer the body fluid collected in the body fluid extracting unit is fed to the test unit and the discarding body fluid after being tested at the test unit is automatically collected or discarded to the discarding body fluid storage unit by operating the body fluid collecting mechanism, so that the test of the body fluid can be automated and the time required for the test can be further reduced.

The body fluid analyzer preferably includes a test unit having glucose oxidase or glucose dehydrogenating enzyme as enzyme. If glucose oxidase or glucose dehydrogenating enzyme is used for the enzyme, the amount (concentration) of glucose in the body fluid can be measured, and the test result can be used for diabetes test, or the like.

The body fluid analyzer preferably includes a test unit having glucose oxidase or glucose dehydrogenating enzyme as enzyme, and a test unit having lysine oxidase as enzyme. If lysine oxidase is used for some enzymes and glucose oxidase or glucose dehydrogenating enzyme is used for other enzymes, the amount of lysine can be detected by the lysine oxidase so that the measurement accuracy of glucose can be enhanced by measuring lysine as a correcting substance.

The body fluid analyzer further preferably includes a medicine supplying mechanism for supplying the body fluid discharge promoting medicine stored in the body fluid discharge promoting medicine storage unit to the body fluid extracting unit; a discarding body fluid storage unit for discarding the body fluid after the test; and a body fluid collecting mechanism for collecting the body fluid collected in the body fluid extracting unit, and discarding the body fluid after the test by the test unit to the discarding body fluid storage unit. According to such body fluid analyzer, the operation of supplying the body fluid discharge promoting medicine to the body fluid extracting unit, the operation of discharging the body fluid discharge promoting medicine in the body fluid extracting unit, the operation of discarding the body fluid collected in the body fluid extracting unit to the discarding body fluid storage unit after testing in the test unit, and the like can be automated so that even unfamiliar subjects can easily test the body fluid himself/herself. The test can be repeatedly carried with the body fluid analyzer remained attached to the arm.

In the body fluid analyzer, the body fluid collecting passage also preferably serves as the medicine collecting passage, the discarding body fluid storage unit also preferably serves as the discarding medicine storage unit, and the medicine collecting mechanism preferably uses the medicine supplying mechanism. According to such body fluid analyzer, the medicine collecting mechanism does not need to be arranged separate from the medicine supplying mechanism, the medicine collecting passage does not need to be arranged separate from the body fluid collecting passage, and the discarding medicine storage unit does not need to be arranged separate from the discarding body fluid storage unit, and hence the structure of the body fluid analyzer can be simplified and the cost can be reduced.

In the body fluid analyzer, the body fluid collecting mechanism preferably uses the medicine supplying mechanism. According to such body fluid analyzer, the body fluid collecting mechanism does not need to be arranged separate from the medicine supplying mechanism, and hence the structure of the body fluid analyzer can be simplified and the cost can be reduced.

The body fluid analyzer further preferably includes a body fluid collecting chip for attaching to a body fluid discharge site; and an analyzer main body to be stationed; wherein the body fluid collecting chip includes the body fluid extracting unit and the test unit; and the analyzer main body includes the medicine supplying mechanism, the medicine collecting mechanism, the body fluid collecting mechanism, and the discarding body fluid storage unit. According to such embodiment, the body fluid collecting chip to be attached to the subject is smaller and lighter so that the attachment feeling of the body fluid analyzer becomes satisfactory.

The body fluid analyzer further preferably includes an analyzer main body for attaching to a body fluid discharge site; and a body fluid collecting chip removably attached to the analyzer main body; wherein the body fluid collecting chip includes the body fluid extracting unit and the test unit; and the analyzer main body includes the medicine supplying mechanism, the medicine collecting mechanism, the body fluid collecting mechanism, and the discarding body fluid storage unit. According to the body fluid analyzer, the body fluid collecting chip can be taken out from the analyzer main body to be replaced with the analyzer main body attached to the subject, so that the test can be continuously carried out.

In the body fluid analyzer, the body fluid extracting unit, the medicine supplying mechanism, the medicine collecting mechanism, the test unit, the body fluid collecting mechanism, and the discarding body fluid storage unit are preferably arranged on the body fluid collecting chip for attaching to the body fluid discharge site. According to such body fluid analyzer, the structure of the body fluid analyzer can be simplified since the body fluid analyzer is integrated.

### BRIEF DESCRIPTION OF THE DRAWINGS

FIG. 1A is a schematic cross-sectional view of an analyzing sensor disclosed in Japanese Unexamined Patent Publication No. 9-5296 (patent document 1);

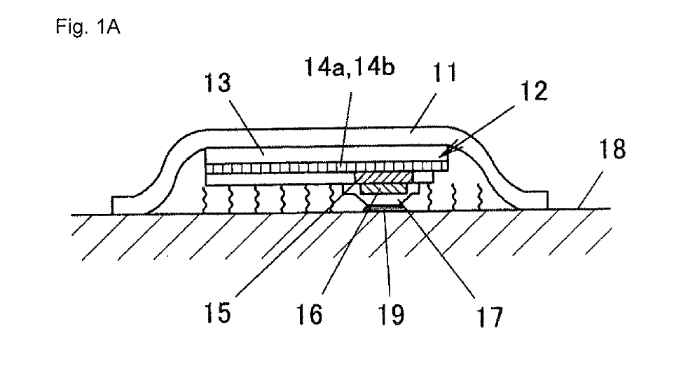

FIG. 1B is a bottom view showing a structure of a biosensor chip of the analyzing sensor disclosed in patent document 1;

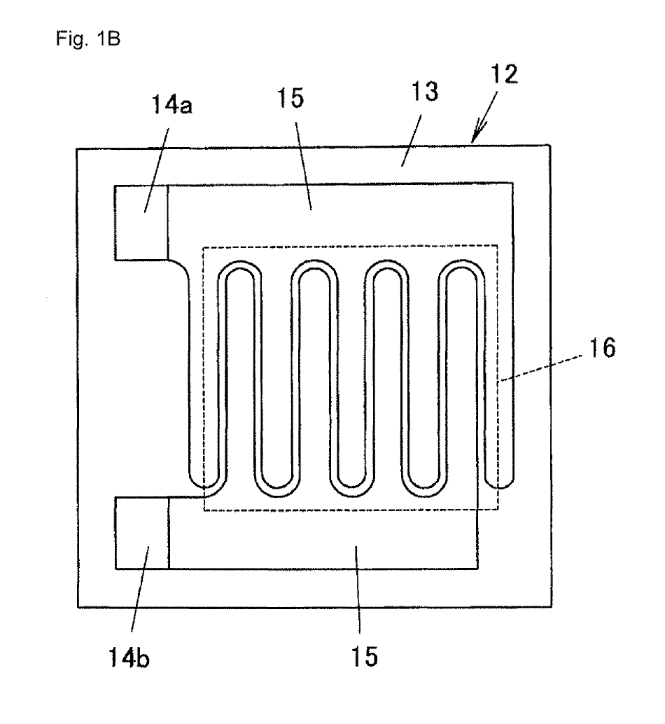

FIG. 2 is a schematic cross-sectional view showing a configuration of a body fluid analyzer according to the first embodiment;

FIG. 3 is a view showing a configuration for supplying and collecting body fluid discharge promoting medicine, calibration fluid, or the like in the body fluid analyzer according to the first embodiment;

FIG. 4 is a plan view of a body fluid collecting chip;

FIG. 5A is a schematic cross-sectional view showing in an enlarged manner a body fluid extracting unit in which the inner surface is inclined;

FIG. 5B is a view describing the operation of the body fluid extracting unit of FIG. 5A;

FIG. 6A is a schematic cross-sectional view showing in an enlarged manner a body fluid extracting unit having a shape different from the body fluid extracting unit of FIG. 5A;

FIG. 6B is a view describing the operation of the body fluid extracting unit of FIG. 6A;

FIG. 7 is a schematic view showing one example of a structure of a test unit;

FIG. 8 is a schematic view showing another example of the structure of the test unit;

FIG. 9 is a plan view showing a body fluid collecting chip of another structure;

FIG. 10A is a view describing a step of conducting the test using the body fluid analyzer according to the first embodiment, and is a cross-sectional view showing the process of supplying the body fluid discharge promoting medicine;

FIG. 10B is a view describing a step of conducting the test using the body fluid analyzer according to the first embodiment, and is a cross-sectional view showing the process of discharging the body fluid discharge promoting medicine after the process of FIG. 10A;

FIG. 10C is a view describing a step of conducting the test using the body fluid analyzer according to the first embodiment, and is a cross-sectional view showing the process of discharging the body fluid discharge promoting medicine and emptying the body fluid extracting unit after the process of FIG. 10B;

FIG. 11A is a view describing a step of conducting a test using the body fluid analyzer according to the first embodiment, and is a cross-sectional view showing the process of collecting the body fluid after the process of FIG. 10C;

FIG. 11B is a view describing a step of conducting a test using the body fluid analyzer according to the first embodiment, and is a cross-sectional view showing the process of conducting the test of the collected body fluid in the test unit after the process of FIG. 11A;

FIG. 11C is a view describing a step of conducting a test using the body fluid analyzer according to the first embodiment, and is a cross-sectional view showing the process of discharging the body fluid after the test is finished and emptying the body fluid extracting unit after the process of FIG. 11B;

FIG. 12 is a schematic cross-sectional view showing a configuration of a body fluid analyzer according to a second embodiment;

FIG. 13 is a schematic cross-sectional view showing a configuration of a body fluid analyzer according to a third embodiment;

FIG. 14 is a view showing a configuration of supplying and discarding the body fluid promoting medicine, or discarding the body fluid in the body fluid analyzer according to the third embodiment;

FIG. 15 is a perspective view showing a state in which the body fluid analyzer of the third embodiment is attached to the arm;

FIG. 16 is a cross-sectional view showing a different configuration of one administration electrode for administering the body fluid discharge promoting medicine;

FIG. 17 is a cross-sectional view showing another further configuration of one administration electrode for administering the body fluid discharge promoting medicine;

FIG. 18 is a cross-sectional view showing another further configuration of one administration electrode for administering the body fluid discharge promoting medicine;

FIG. 19 is a cross-sectional view showing a different configuration of the other administration electrode for administering the body fluid discharge promoting medicine;

FIG. 20 is a cross-sectional view showing another further configuration of the other administration electrode for administering the body fluid discharge promoting medicine;

FIG. 21 is a perspective view showing a body fluid analyzer according to a fourth embodiment;

FIG. 22 is a schematic cross-sectional view showing a configuration of the body fluid analyzer according to the fourth embodiment; and

FIG. 23 is a schematic cross-sectional view showing a configuration of a body fluid analyzer according to the fifth embodiment.

### BEST MODE FOR CARRYING OUT THE INVENTION

Preferred embodiments of the present invention will be described below with reference to the accompanied drawings.

### First Embodiment

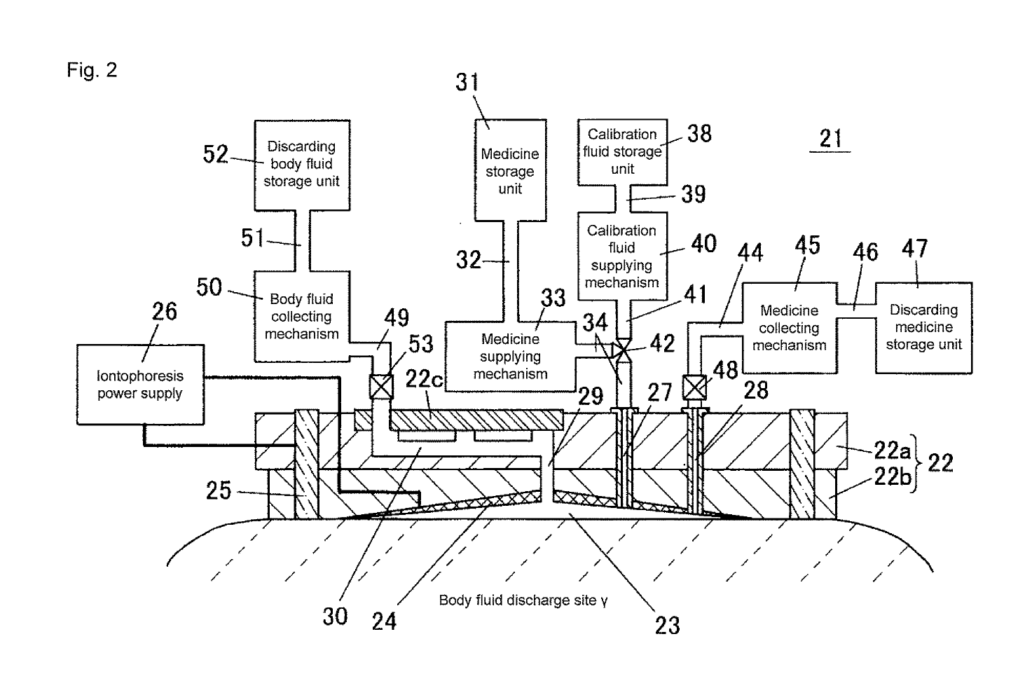

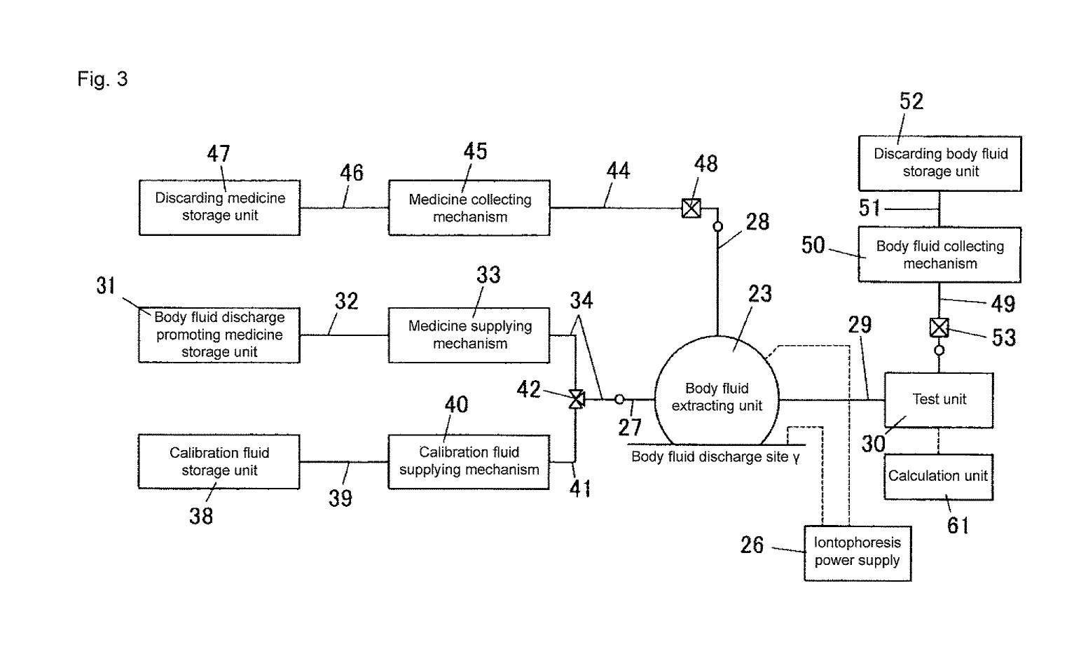

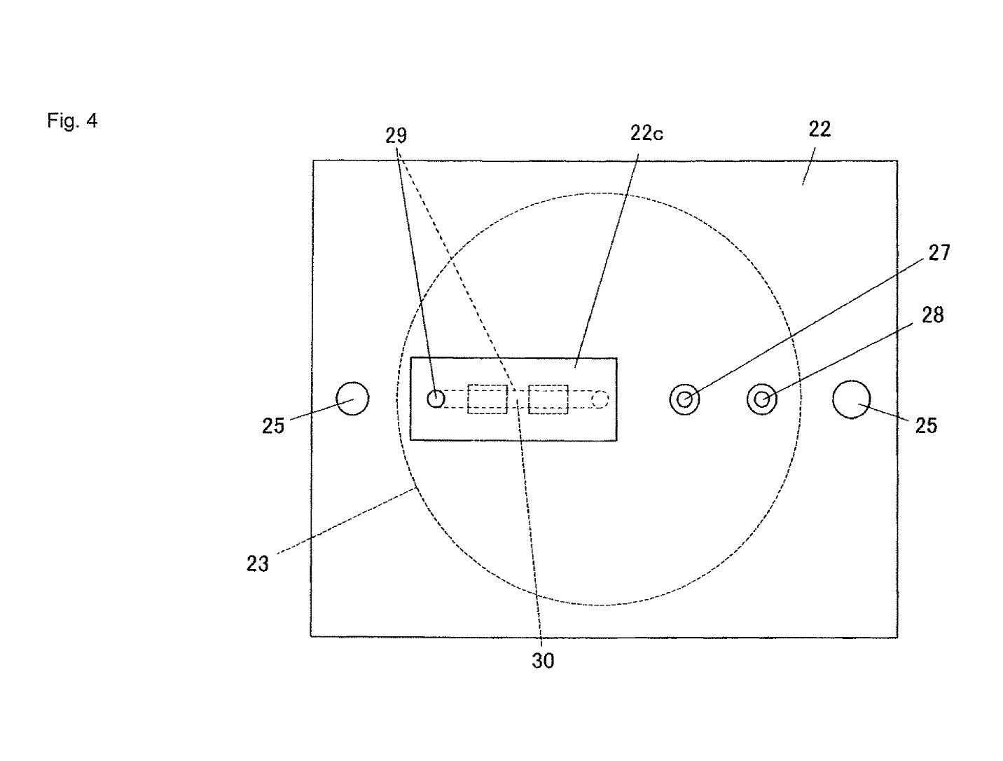

FIG. 2 is a schematic cross-sectional view showing a configuration of a body fluid analyzer 21 according to a first embodiment. FIG. 3 is a view showing a configuration for supplying and collecting body fluid discharge promoting medicine and calibration fluid in the body fluid analyzer 21. FIG. 4 is a plan view of a body fluid collecting chip 22 used in the body fluid analyzer 21.

The body fluid analyzer 21 according to the first embodiment is mainly configured by a body fluid collecting unit for collecting and testing the body fluid, and a mechanism portion for supplying and collecting the body fluid discharge promoting medicine, the calibration fluid or the like to the body fluid collecting unit, and discarding the tested body fluid.

Sweat is the easiest and most convenient body fluid to be collected, and a case of collecting sweat will be hereinafter described, but it should be recognized that the body fluid may be cell fluid of the subcutaneous tissue, tissue fluid of the living tissue, or the like.

(Description of Body Fluid Collecting Unit)

First, the body fluid collecting unit will be described.

With reference to FIG. 2, the body fluid collecting unit includes the body fluid collecting chip 22 as a mother body. The body fluid collecting chip 22 is obtained by stacking and integrating an upper plate 22a and a lower plate 22b made from an insulating material such as plastic or glass. In particular, the body fluid collecting chip 22 desirably has an appropriate flexibility so as to curve along a body fluid discharge site γ. A body fluid extracting unit 23 for holding the body fluid discharge promoting medicine and collecting the body fluid extracted from the body fluid discharge site γ is arranged at the lower surface of the body fluid collecting chip 22. The body fluid extracting unit 23 includes a depression which inner wall surface (upper surface) is gradually inclined. For instance, the body fluid extracting unit 23 is formed to a depression having a conical shape or a triangular groove shape.

One administration electrode 24 is arranged on the inner wall surface of the body fluid extracting unit 23 by a conductive film such as metal. The other administration electrode 25 is embedded at a position distant from the body fluid extracting unit 23 or the administration electrode 24 of the body fluid collecting chip 22. The lower end face of the administration electrode 25 is exposed at the lower surface of the body fluid collecting chip 22. The administration electrodes 24, 25 are electrically connected to an output terminal of an iontophoresis power supply 26. The iontophoresis power supply 26 can apply voltage between the administration electrodes 24, 25

Various types of iontophoresis power supplies 26 can be used. In other words, the iontophoresis power supply 26 to be used may be a power supply of a continuous DC type in which the DC current is continuously flowed, a pulse DC type in which the DC current is intermittently flowed, a pulse depolarization DC type in which the stimulation of the skin is small, an AC type in which the AC current is flowed, or the like.

The body fluid collecting chip 22 is perforated with a medicine injecting hole 27 and a medicine collecting hole 28, which pass from the upper surface to the lower surface. The lower end of the medicine injecting hole 27 and the lower end of the medicine collecting hole 28 are both opened in the body fluid extracting unit 23.

A body fluid feeding path 29 for feeding the collected body fluid in the body fluid extracting unit 23 is arranged in the body fluid collecting chip 22. The body fluid feeding path 29 extends to the upper side from the top of the body fluid extracting unit 23, and then extends horizontally in the body fluid collecting chip 22, and again extends to the upper side.

As shown in FIG. 2 and FIG. 4, a test unit 30 for testing a specific component contained in the body fluid is arranged in a region in the body fluid feeding path 29 that extends horizontally. An opening is formed in the upper plate 22a at the upper surface of the test unit 30, and a test unit cover 22c is removably fitted to the opening. The body fluid feeding path 29 is passed through the test unit 30, and then passed vertically through the test unit cover 22c to open at the upper surface of the test unit cover 22c.

(Description of Body Fluid Extracting Unit)

The structure of the body fluid extracting unit 23 will now be described.

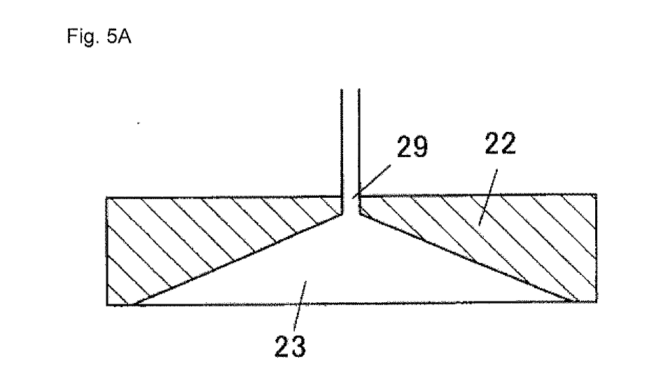

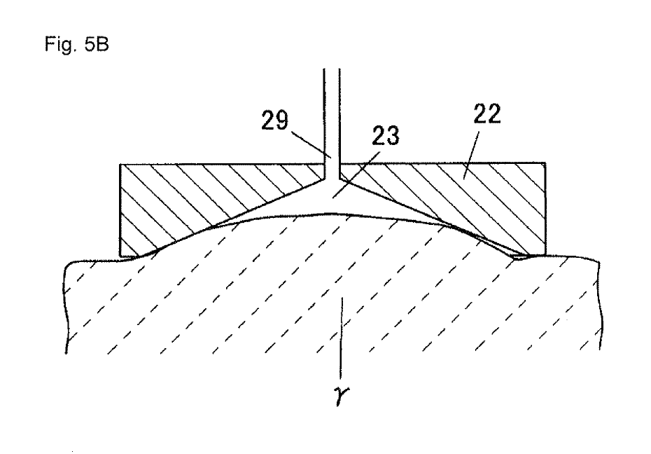

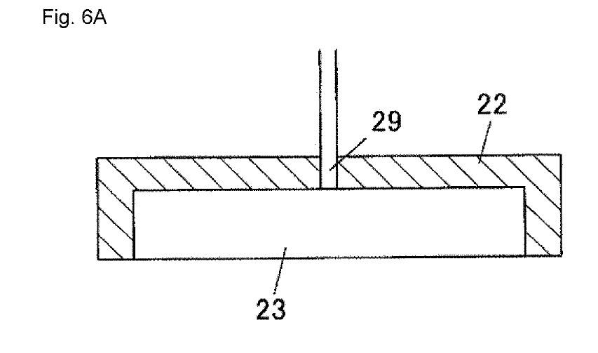

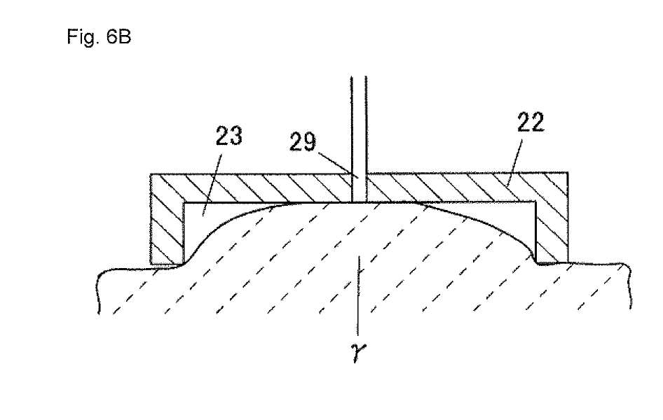

A body fluid extracting unit 23 having a shape different from the body fluid extracting unit 23 (FIG. 5A) arranged in the body fluid analyzer 21 according to the first embodiment is shown in FIG. 6A. The body fluid extracting unit 23 having a columnar shape or a rectangular column shape (i.e., square cross-section) is arranged in the body fluid collecting chip 22 shown in FIG. 6A. To prevent the body fluid discharge promoting medicine in the body fluid extracting unit 23 from leaking out when extracting the body fluid by the body fluid discharge promoting medicine, the body fluid collecting chip 22 needs to be pressed against the body fluid discharge site γ (skin) with a pressure of a certain extent. Thus, in the body fluid extracting unit 23 of the shape shown in FIG. 6A, when the body fluid collecting chip 22 is pressed against the body fluid discharge site γ of the body, the body fluid discharge site γ (skin) rises as shown in FIG. 6B thus entering the body fluid extracting unit 23, which may block the body fluid feeding path 29 for collecting the body fluid.

In the body fluid collecting chip 22 of the present embodiment, on the other hand, the inner wall surface of the body fluid extracting unit 23 is gradually inclined to the periphery or both sides of the body fluid feeding path 29, and the body fluid feeding path 29 is arranged at the top of the body fluid extracting unit 23, as shown in FIG. 5A. Therefore, even if the body fluid collecting chip 22 is pressed against the body fluid discharge site γ of the subject and the body fluid discharge site γ enters the body fluid extracting unit 23, the body fluid feeding path 29 is less likely to be blocked, and the collection of the body fluid is less likely to be inhibited as in FIG. 5B.

If the inner wall surface of the body fluid extracting unit 23 is gradually inclined, the internal capacity of the body fluid extracting unit 23 can be reduced, and the injection amount (consumption amount) of the body fluid discharge promoting medicine can be reduced. Furthermore, since the internal capacity of the body fluid extracting unit 23 can be reduced, the arriving speed of the body fluid to the test unit 30 is increased when the body fluid collected in the body fluid extracting unit 23 is fed to the test unit 30 thus shortening the measurement time.

(Description of Test Unit)

The structure of the test unit 30 will be specifically described.

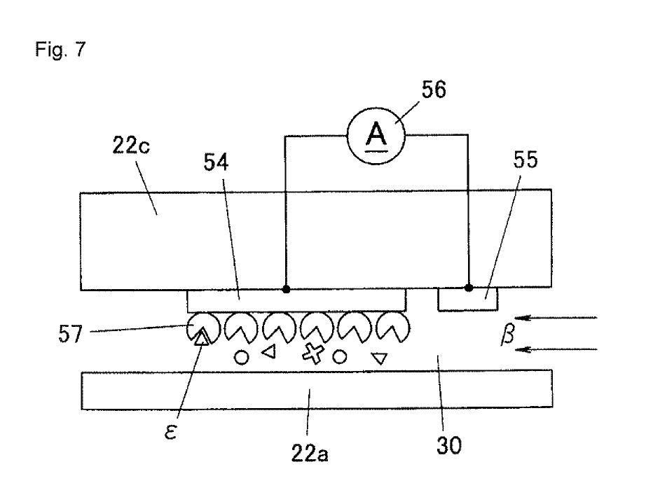

With reference to FIG. 7, the test unit 30 includes the cover 22c made of insulating material, where a pair of test electrodes 54, 55 is arranged at the lower surface thereof. An ammeter 56 is connected between the test electrodes 54, 55.

When conducting the test, the test unit cover 22c is separated from the body fluid collecting chip 22, an enzyme 57 that specifically reacts with a specific component (e.g., glucose) to be tested is immobilized on the surface of one test electrode 54, and the test unit cover 22c is again attached to the opening of the upper plate 22a. As a result, the enzyme 57 is positioned on the upper surface of the test unit 30. The body fluid β collected in the body fluid extracting unit 23 is fed to the test unit 30 through the body fluid feeding path 29. In this case, if a specific component ε is contained in the body fluid β, an oxidation-reduction reaction occurs between the specific component ε and the enzyme 57 thus flowing current between the test electrodes 54, 55, and such current is detected by the ammeter 56. A calculation unit 61 (see FIG. 3) configured by an electronic circuit determines whether or not the specific component ε is contained in the body fluid β based on the current value. Alternatively, the concentration of the specific component ε contained in the body fluid β is calculated, and the result is displayed on the display unit.

The method of electrochemically measuring the reaction amount of the specific component ε and the enzyme 57 includes a method of using an oxygen electrode, a method of using a hydrogen peroxide electrode, and a method of using a mediator enzyme electrode. In the method of using the oxygen electrode, the test electrode 54 is assumed as a platinum electrode and the test electrode 55 is assumed as a silver electrode. When the amount of specific component ε increases, the consumption amount of oxygen increases by the enzyme reaction, so that the dissolved oxygen amount in the body fluid decreases. In the oxygen electrode, the current proportional to the dissolved oxygen amount flows, so that the specific component ε can be detected by measuring the current value or the amount thereof can be measured.

When using the hydrogen peroxide electrode, if the amount of specific component ε increases, the generation amount of hydrogen peroxide increases by the enzyme reaction, so that the current value between the test electrodes 54, 55 increases. Therefore, the current value becomes greater as the amount of specific component ε increases, whereby the amount of specific component ε can be measured by measuring the current value.

When using the mediator enzyme electrode, the electrons generated by the enzyme reaction of the specific component ε are detected by the test electrodes 54, 55 through the mediator (film that promotes electron migration between the electrode and the enzyme). The electron to be transmitted increases as the amount of specific component ε increases, and the current between the test electrodes 54, 55 increases, and hence the amount of specific component ε can be measured by measuring the current value.

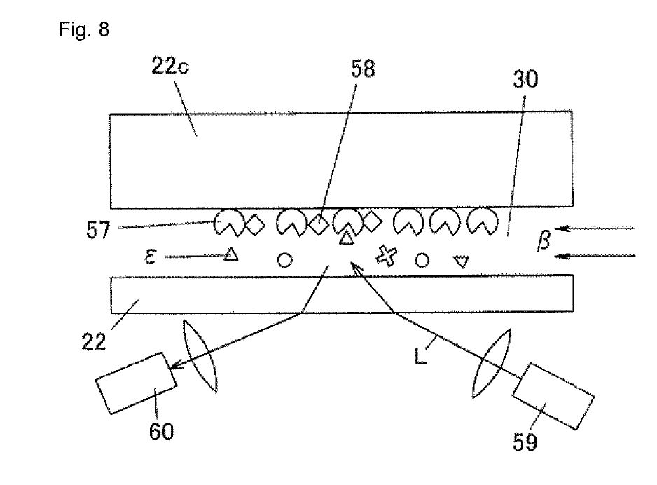

FIG. 8 shows another example of a specific structure of the test unit 30. The test unit 30 of FIG. 8 is an optical type, where the enzyme 57 that specifically reacts with the specific component ε, the color forming dye 58, and the enzyme (peroxidase) that becomes a catalyst with respect to the hydrogen peroxide are immobilized on the lower surface of the test unit cover 22c. At least the portion on the lower side of the test unit of the body fluid collecting chip 22 is formed by a transparent material, and a light projecting unit 59 and a light receiving unit 60 are arranged on the lower side of the body fluid collecting chip 22.

In the test unit 30, when the body fluid β is supplied, the hydrogen peroxide is generated by the reaction of the specific component ε and the enzyme 57. Furthermore, active oxygen is generated from the hydrogen peroxide with the action of the enzyme that becomes a catalyst with respect to the hydrogen peroxide. The optical property (wavelength or intensity) in the test unit 30 changes by the color reaction of the active oxygen and the color forming dye 58. Therefore, the specific component ε in the body fluid is detected or the amount thereof is measured by irradiating the test unit 30 with white light L from the light projecting unit 59, receiving the light reflected at the test unit 30 with the light receiving unit 60, and analyzing the light spectrum of the reflected light.

The enzyme is used herein to detect the specific component in the body fluid, but in addition, reagent or the like may be used.

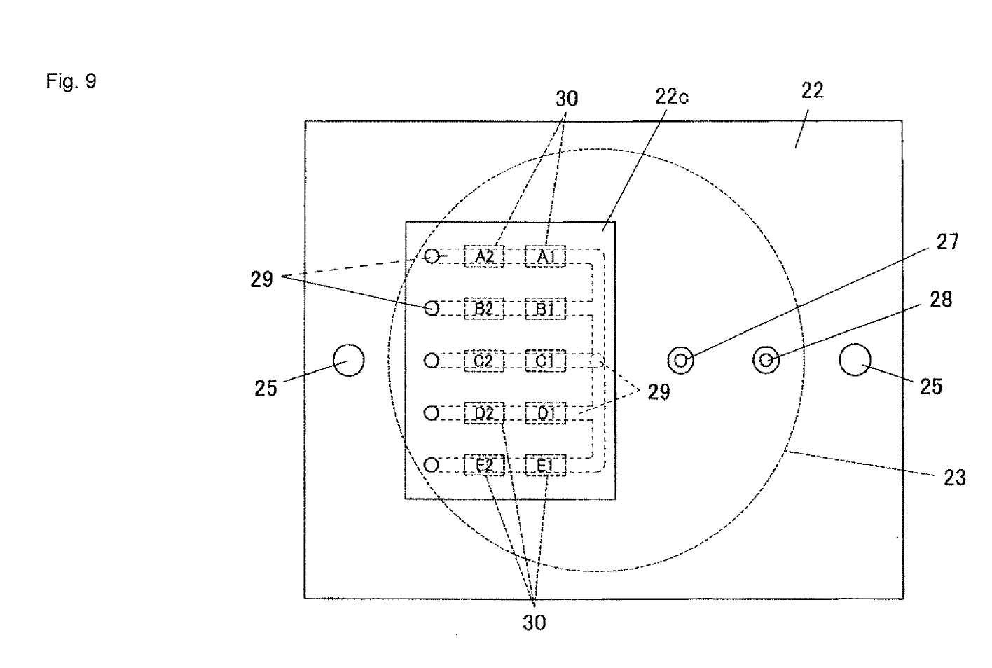

FIG. 4 shows the body fluid collecting chip 22 including one test unit 30, but a plurality of test units 30 may be arranged to an array form. FIG. 9 shows one example of the body fluid collecting chip 22 including a plurality of test units 30, as another configuration. In the configuration of FIG. 9, the horizontally extending region of the body fluid feeding path 29 is branched into plurals, and a plurality of test units 30 is arranged in each body fluid feeding path 29. In the configuration of FIG. 9, the body fluid feeding path 29 is branched into five and two test units 30 are arranged in each body fluid feeding path, but is not limited to such number. In the configuration of FIG. 9, a plurality of body fluid feeding paths 29 is opened at the upper surface of the test unit cover 22c, but the body fluid feeding paths 29 are collected to one at the upper side of the test unit cover 22c (or inside the test unit cover 22c).

The plurality of test units 30 arranged to an array form are each immobilized with an enzyme A1, A2, B1, B2, C1, C2, . . . . In a first mode, the enzymes A1, A2, B1, B2, C1, C2, . . . may all be different types of enzymes. According to such mode, different specific components can be tested with each enzyme A1, A2, B1, B2, C1, C2, . . . , a plurality of specific components can be measured at the same time (at once), and the test can be efficiently carried out.

In a second mode, the enzymes A1, A2, B1, B2, C1, C2, . . . may all be the same type of enzymes. According to such mode, the same specific component can be tested with each enzyme A1, A2, B1, B2, C1, C2, . . . , so that one specific component can be measured over plural times all at once, thereby increasing the accuracy of the measurement accuracy.

In a third mode, some of the enzymes are the same type of enzymes, and some are different types of enzymes. For instance, if the test units 30 belonging to the same body fluid feeding path 29 are assumed as one set, the enzyme of the test units 30 in the one set may be different from each other and the combination of enzymes may be the same among different sets. In relation to FIG. 9, the enzymes A1, B1, C1, . . . are the same enzymes, the enzymes A2, B2, C2, . . . are the same enzymes, and the enzymes A1, B1, C1, . . . and the enzymes A2, B2, C2, . . . are differed. Alternatively, the enzymes (e.g., enzyme A1 and enzyme A2) positioned in the same body fluid feeding path 29 may be the same enzyme, and the enzymes belonging to different body fluid feeding paths 29 may be enzymes different from each other. According to such mode, the advantage of the first mode and the advantage of the second mode can be obtained, so that a plurality of specific components can be measured in one time and the same specific component can be measured over plural times, whereby the measurement task becomes more efficient and the measurement accuracy becomes higher.

(Description of Mechanism Portion for Supplying and Collecting Fluid)

The mechanism portion for supplying and collecting the body fluid discharge promoting medicine α and the calibration fluid to the body fluid extracting unit 23, and discarding the body fluid β after the test will now be described with reference to FIG. 2 and FIG. 3.

A body fluid discharge promoting medicine storage unit 31 holds the body fluid discharge promoting medicine. Medicine containing pilocarpine or acetylcholine is used for the body fluid discharge promoting medicine. The perspiration from under the skin or inside the body can be promoted and the necessary amount of body fluid (sweat) can be collected in a short time by using the body fluid discharge promoting medicine. The body fluid discharge promoting medicine storage unit 31 is connected to a medicine supplying mechanism 33 by a medicine flow path 32, and the medicine supplying mechanism 33 is in turn connected to the medicine injecting hole 27 by the medicine flow path 34. The medicine flow path 34 includes a switching valve 42 for opening and closing the medicine flow path 34.

The medicine injection passage for supplying the body fluid discharge promoting medicine to the body fluid extracting unit 23 includes the medicine flow paths 32, 34, and the medicine injecting hole 27. The medicine supplying mechanism 33 includes a small pump, where the body fluid discharge promoting medicine stored in the body fluid discharge promoting medicine storage unit 31 can be supplied into the body fluid extracting unit 23 through the medicine injection passage by driving the medicine supplying mechanism 33.

The body fluid discharge promoting medicine storage unit 31 is removable, and the body fluid discharge promoting medicine storage unit 31 can be detached to supplement the body fluid discharge promoting medicine when the body fluid discharge promoting medicine runs out.

A calibration fluid storage unit 38 holds the calibration fluid for calibrating the measurement value of the test unit 30. The calibration fluid storage unit 38 is connected to a calibration fluid supplying mechanism 40 by a calibration fluid flow path 39, and in turn, the calibration fluid supplying mechanism 40 is connected to a switching valve 42 by the calibration fluid flow path 41.

The calibration fluid feeding passage for supplying the calibration fluid to the test unit 30 includes the calibration fluid flow paths 39, 41, the medicine injecting hole 27, and the body fluid feeding path 29. The calibration fluid supplying mechanism 40 includes a small pump, where the calibration fluid stored in the calibration fluid storage unit 38 can be fed to the test unit 30 through the calibration fluid feeding passage by driving the calibration fluid supplying mechanism 40. The reliability of the test result can be enhanced by calibrating the test unit 30 using the calibration fluid before actually performing the test of the body fluid or periodically.

The calibration fluid storage unit 38 is removable, and the calibration fluid storage unit 38 can be detached to supplement the calibration fluid when the calibration fluid runs out.

The switching valve 42 (three way valve) can be switched to a state in which the medicine injecting hole 27 side and the medicine supplying mechanism 33 side are communicated and the calibration fluid supplying mechanism 40 side is closed, a state in which the medicine injecting hole 27 side and the calibration fluid supplying mechanism 40 side are communicated and the medicine supplying mechanism 33 side is closed, and a state in which the medicine injecting hole 27 side is closed. The body fluid discharge promoting medicine can be supplied to the body fluid extracting unit 23 by switching the switching valve 42 to the first state, and the calibration fluid can be fed to the test unit 30 by switching the switching valve 42 to the second state. When collecting the body fluid discharge promoting medicine and the body fluid, the body fluid discharge promoting medicine in the body fluid discharge promoting medicine storage unit 31 and the calibration fluid in the calibration fluid storage unit 38 can be prevented from being drawn out by switching the switching valve 42 to the third state.

The medicine collecting hole 28 is connected with a discarding medicine flow path 44 including a open/close valve 48, and the other end of the discarding medicine flow path 44 is connected to a medicine collecting mechanism 45. Furthermore, the medicine collecting mechanism 45 is connected to a discarding medicine storage unit 47 by a discarding medicine flow path 46. The discarding medicine storage unit 47 is a container for collecting and storing the extra used body fluid discharge promoting medicine (hereinafter sometimes referred to as discarding medicine).

The medicine collecting passage for collecting the discarding medicine includes a medicine collecting hole 28 and the discarding medicine flow paths 44, 46. The medicine collecting mechanism 45 is a small pump, and collects or discards the extra discarding medicine remaining in the body fluid extracting unit 23 to the discarding medicine storage unit 47 through the medicine collecting passage by driving the medicine collecting mechanism 45. The open/close valve 48 closes the discarding medicine flow path 44 to prevent the body fluid discharge promoting medicine from entering the medicine collecting passage when being supplied to the body fluid extracting unit 23, or the discarding medicine from being drawn out when collecting the body fluid.

The discarding medicine storage unit 47 is removable, where the discarding medicine storage unit 47 can be detached to discard the discarding medicine when the storage unit becomes full with the discarding medicine.

The discarding body fluid flow path 49 including an open/close valve 53 is connected to the terminating end of the body fluid feeding path 29, and the other end of the discarding body fluid flow path 49 is connected to the body fluid collecting mechanism 50. Moreover, the body fluid collecting mechanism 50 is connected to the discarding body fluid storage unit 52 by the discarding body fluid flow path 51. The discarding body fluid storage unit 52 is a container for discarding the tested body fluid (hereinafter sometimes referred to as discarding body fluid).

The body fluid collecting passage for collecting the discarding body fluid includes the body fluid feeding path 29 and the discarding body fluid flow paths 49, 51. The body fluid collecting mechanism 50 is a small pump, and collects or discards the discarding medicine that passed the test unit 30 to the discarding body fluid storage unit 52 through the body fluid collecting passage by driving the body fluid collecting mechanism 50. The open/close valve 53 closes the discarding body fluid flow path 49 to prevent the body fluid discharge promoting medicine from entering the test unit 30 when being supplied to the body fluid extracting unit 23, or the discarding body fluid in the discarding body fluid storage unit 52 from being drawn out when collecting the discarding medicine.

The discarding body fluid storage unit 52 is removable, where the discarding body fluid storage unit 52 can be detached to discard the discarding body fluid when the storage unit becomes full with the discarding body fluid.

(Testing Method)

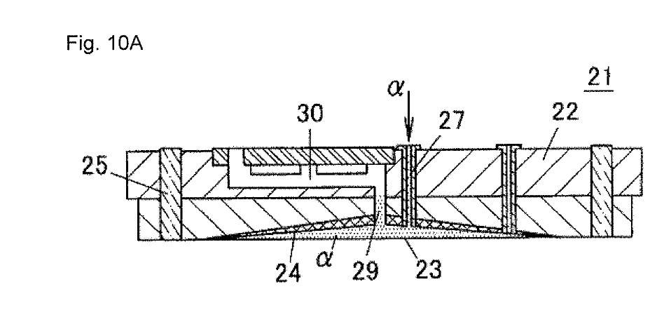

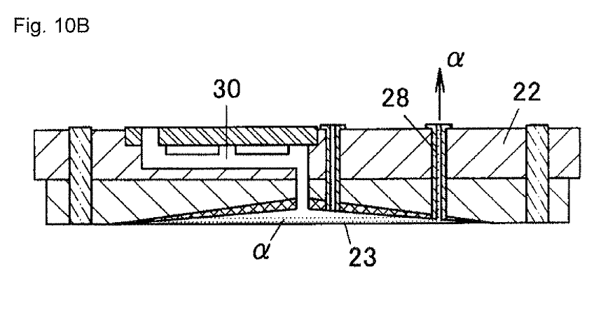

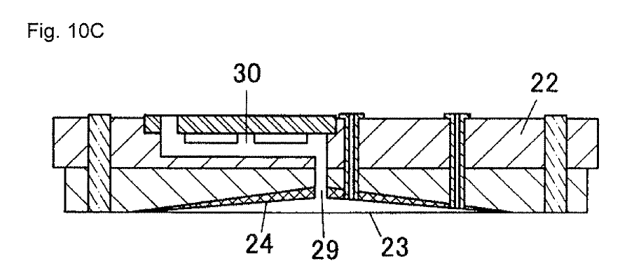

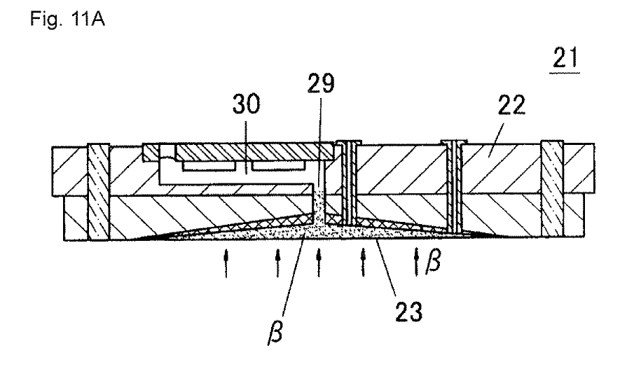

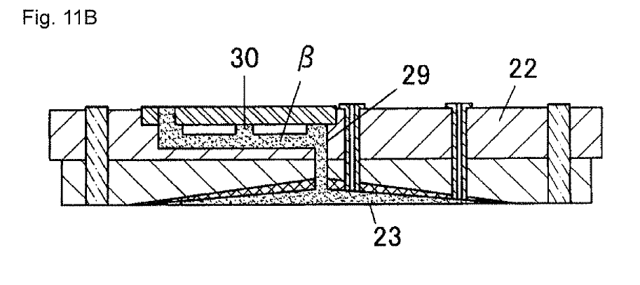

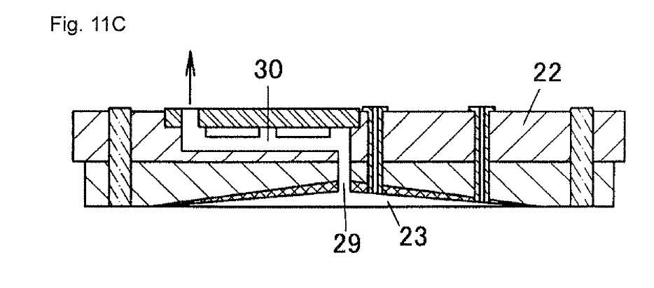

The process of performing the test of illnesses such as diabetes using the body fluid analyzer 21 configured as above will now be described. FIGS. 10A to 10C and FIGS. 11A to 11C are cross-sectional views showing one part of the process.

When performing the test, the body fluid analyzer 21 is first attached to the body fluid discharge site γ (e.g., certain portion of wrist or arm) of the subject. In this case, the body fluid analyzer 21 is attached so that the lower surface of the body fluid collecting chip 22 (surface arranged with body fluid extracting unit 23) is closely attached to the body fluid discharge site γ. The administration electrode 25 is also brought into contact with the body fluid discharge site γ or the vicinity thereof.

If calibration of the test unit 30 needs to be carried out prior to the test, the open/close valves 48, 53 are closed, the switching valve 42 is switched to the state in which the calibration fluid supplying mechanism 40 side and the medicine injecting hole 27 side are communicated, and then the calibration fluid supplying mechanism 40 is operated so that the calibration fluid is fed from the calibration fluid storage unit 38. The calibration fluid is fed to the body fluid extracting unit 23 through the calibration fluid flow paths 39, 41 and the medicine injecting hole 27, and then to the test unit 30 through the body fluid feeding path 29. When the body fluid extracting unit 23 and the test unit 30 are filled with the calibration fluid, the calibration fluid supplying mechanism 40 is stopped and the switching valve 42 is closed. The output value of the test unit 30 then can be calibrated while looking at the output value of the test unit 30.

After the calibration of the test unit 30 is terminated, the open/close valve 53 is opened, the body fluid collecting mechanism 50 is operated, and the calibration fluid in the test unit 30 and the body fluid extracting unit 23 is aspirated. The calibration fluid is thereby discarded to the discarding body fluid storage unit 52, so that the body fluid extracting unit 23 and the test unit 30 become empty. The body fluid collecting mechanism 50 is stopped and the open/close valve 53 is closed after the body fluid extracting unit 23 and the test unit 30 become empty.

The measurement accuracy of the body fluid analyzer 21 can be increased by calibrating the test unit 30 in advance in such manner.

As shown in FIG. 10A, when the test is started, the body fluid discharge promoting medicine α is fed from the medicine injecting hole 27 to the body fluid extracting unit 23, so that the body fluid extracting unit 23 is filled with the body fluid discharge promoting medicine α. In other words, the switching valve 42 is switched to a state in which the medicine supplying mechanism 33 side and the medicine injecting hole 27 side are communicated with the open/close valves 48, 53 closed, and then the medicine supplying mechanism 33 is operated so that the body fluid discharge promoting medicine α is fed from the body fluid discharge promoting medicine storage unit 31. The body fluid discharge promoting medicine α is fed to the body fluid extracting unit 23 through the medicine flow paths 32, 34 and the medicine injecting hole 27. The medicine supplying mechanism 33 is stopped and the switching valve 42 is closed after the body fluid extracting unit 23 is filled with the body fluid discharge promoting medicine α.

This state is maintained for a while, so that the body fluid discharge promoting medicine α held in the body fluid extracting unit 23 penetrates to the body fluid discharge site γ to be percutaneously administered. The body fluid discharge promoting medicine α that contains pilocarpine or acetylcholine promotes perspiration from under the skin or inside the body, and hence the body fluid collecting speed becomes faster by using the body fluid discharge promoting medicine α than with the natural perspiration, and the body fluid collecting time can be reduced.

Generally, the passive diffusion is dominant in the body fluid discharge promoting medicine which molecular mass is smaller than or equal to 200, and such medicine can be passively absorbed to the skin by being brought into contact with or by being applied to the skin surface. However, the passive diffusion is difficult for the body fluid discharge promoting medicine which molecular mass is greater than or equal to 200, and a physical method needs to be considered to promote the absorption. As there is a limit in improving the medicine transmissivity with only the passive diffusion, the electrochemical potential of the ionic drug needs to be raised by creating a potential difference between the body fluid discharge promoting medicine and the skin, and active absorption and diffusion of the body fluid discharge promoting medicine are needed. Since the stratum corneum demonstrates high electrical resistance when an electrical energy is applied to the skin, the current mainly flows to the sweat gland and other appendicular organs. In this case, if the ionic drug is used for the body fluid discharge promoting medicine, the body fluid discharge promoting medicine may be absorption-diffused to under the skin through the organs.

Therefore, the body fluid discharge promoting medicine α may be passively diffused (spontaneous penetration) to the body fluid discharge site γ without using the iontophoresis power supply 26 and the administration electrodes 24, 25. In the body fluid analyzer 21 according to the present embodiment, however, the iontophoresis power supply 26 and the administration electrodes 24, 25 are arranged, so that the penetration of the body fluid discharge promoting medicine α to under the skin can be further promoted by applying voltage between the administration electrodes 24, 25 by the iontophoresis power supply 26 and flowing current from the body fluid discharge site γ to the body fluid discharge promoting medicine α. The body fluid collecting speed can be increased and the body fluid collecting time can be further reduced.

After a predetermined time necessary for administrating the body fluid discharge promoting medicine α to under the skin has elapsed, the iontophoresis power supply 26 is turned OFF and the body fluid discharge promoting medicine α remaining in the body fluid extracting unit 23 is discharged as shown in FIG. 10B. The open/close valve 48 is then opened and the medicine collecting mechanism 45 is operated so that the body fluid discharge promoting medicine α in the body fluid extracting unit 23 is discharged. The body fluid discharge promoting medicine α in the body fluid extracting unit 23 is thus aspirated and discharged to the discarding medicine storage unit 47. After the discarding medicine is finished being discharged, the medicine collecting mechanism 45 is stopped and the open/close valve 48 is closed.

The test unit 30 and the body fluid extracting unit 23 are empty, as shown in FIG. 10C. Immediately after the body fluid discharge promoting medicine α is discharged, but the body fluid β extracted from the body fluid discharge site γ gradually accumulates in the body fluid extracting unit 23, as shown in FIG. 11A since there is a temporal delay until the perspiration starts from when the body fluid discharge promoting medicine α is administered to under the skin.

The body fluid β and the body fluid discharge promoting medicine α can be avoided from being mixed or the body fluid β can be avoided from being diluted with the body fluid discharge promoting medicine α when the body fluid is collected in the body fluid extracting unit 23, which became empty by discharging the body fluid discharge promoting medicine α, so that the measurement accuracy of a specific component in the body fluid can be enhanced. In particular, the measurement can be carried out even if the specific component has low concentration.

When sufficient body fluid β is accumulated in the body fluid extracting unit 23, the open/close valve 53 is opened and the body fluid collection mechanism 50 is operated. As shown in FIG. 11B, the body fluid β accumulated in the body fluid extracting unit 23 is then moved to the test unit 30 or passed through the test unit 30, and the body fluid β is tested in the test unit 30.

The enzyme 57 that specifically reacts with the specific component in the body fluid is immobilized on the upper surface of the passage of the test unit 30, and the test is conducted through a method described using FIG. 7 or FIG. 8. If glucose oxidase (GOD) or glucose dehydrogenating enzyme (GDH) is used as the enzyme, the amount (concentration) of glucose in the body fluid can be measured and the test result can be used for the diabetes test, or the like.

Two or more test units 30 may be arranged, where the lysine oxidase (LOD) may be immobilized for the enzyme in some test units 30, and the glucose oxidase (GOD) or glucose dehydrogenating enzyme (GDH) may be immobilized for the enzyme in the remaining test units 30. In this case, the amount of lysine can be detected by the lysine oxidase, so that the measurement accuracy of the glucose can be enhanced by measuring the lysine as a correction substance.

The body fluid β (discarding body fluid) used in the measurement is discarded to the discarding body fluid storage unit 52 through the discarding body fluid flow paths 49, 51. When the body fluid β in the body fluid extracting unit 23 and the test unit 30 are discharged and the units become empty, the body fluid collecting mechanism 50 is stopped and the open/close valve 53 is closed.

As a result, the inside of the body fluid collecting chip 22 becomes empty, as shown in FIG. 11C, and returns to the state at the beginning of the starting of the test in which the body fluid discharge promoting medicine α and the body fluid β are not contained. Therefore, the test can be continuously conducted without detaching the body fluid analyzer 21 from the body fluid discharge site γ as long as the function of the enzyme continues. If the sensor function lowers due to the lifespan of the enzyme, or the like, the test unit cover 22c is detached from the body fluid collecting chip 22, and changed to a new test unit cover 22 immobilized with the enzyme so that the test can be repeatedly conducted.

### Variant of First Embodiment

As another structure for supplying the body fluid discharge promoting medicine, the body fluid discharge promoting medicine storage unit 31 may be a breakable liquid container (capsule) in which the body fluid discharge promoting medicine of liquid form is sealed, and the medicine supplying mechanism 33 may be a breaking tool (not shown) for breaking the body fluid discharge promoting medicine storage unit 31. In this case, when the body fluid discharge promoting medicine storage unit 31 is broken by the breaking tool or the medicine supplying mechanism 33, the body fluid discharge promoting medicine in the body fluid discharge promoting medicine storage unit 31 is flowed out to the medicine injecting hole 27 to be supplied to the body fluid extracting unit 23.

A method of efficiently penetrating the body fluid discharge promoting medicine α to the body fluid discharge site γ includes a method of using ultrasonic vibration. For instance, an element for generating an ultrasonic vibration such as a piezoelectric vibrator may be arranged on the inner surface of the body fluid extracting unit 23, and the ultrasonic vibration may be generated between the body fluid discharge promoting medicine α in the body fluid extracting unit 23 and the body fluid discharge site γ. If the ultrasonic vibration is generated after supplying the body fluid discharge promoting medicine α to the body fluid extracting unit 23, the penetration of the body fluid discharge promoting medicine α is promoted by such vibration.

Another medicine collecting mechanism 45 (or method of discharging the body fluid discharge promoting medicine in the body fluid extracting unit 23 from the body fluid extracting unit 23) includes pushing out the body fluid discharge promoting medicine α from the body fluid extracting unit 23 by the pressure of the body fluid β extracted from the body fluid discharge site γ. According to such method, the power is not necessary for the medicine collecting mechanism 45.

Another method of discharging the body fluid discharge promoting medicine in the body fluid extracting unit 23 from the body fluid extracting unit 23 includes feeding a volatile liquid to the body fluid extracting unit 23 and substituting the volatile liquid with the body fluid discharge promoting medicine α or mixing the volatile liquid in the body fluid discharge promoting medicine α, and thereafter, volatilizing the volatile liquid. According to such method, the body fluid extracting unit can be easily emptied by evaporating the volatile liquid.

### Second Embodiment

A body fluid analyzer 66 according to a second embodiment will now be described.

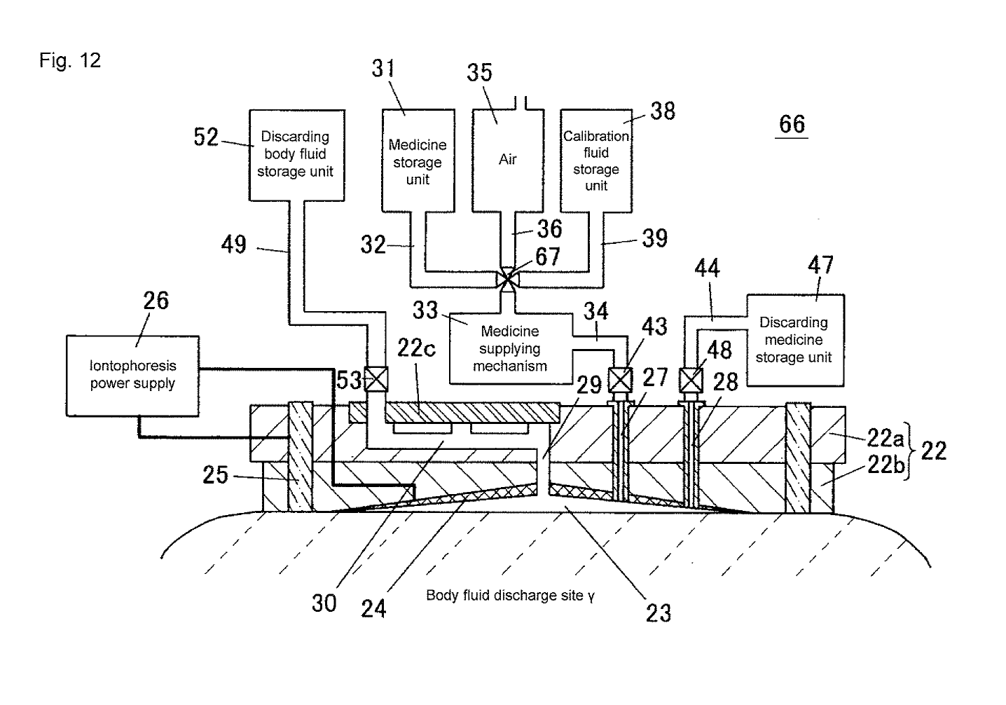

FIG. 12 is a schematic cross-sectional view showing a configuration of a body fluid analyzer 66. In the body fluid analyzer 66, the medicine supplying mechanism 33 in the body fluid analyzer 21 according to the first embodiment also serves as the calibration fluid supplying mechanism 40, the medicine collecting mechanism 45, and the body fluid collecting mechanism 50. The body fluid analyzer 66 has a structure similar to the body fluid analyzer 21, and hence the difference will be mainly described below.

In the body fluid analyzer 66, the discarding body fluid storage unit 52 is connected to the discarding body fluid flow path 49 since the body fluid collecting mechanism 50 is not arranged. Similarly, the discarding medicine storage unit 47 is connected to the discarding medicine flow path 44 since the medicine collecting mechanism 45 is not arranged.

The switching valve 67 is arranged on the medicine flow path 32 connecting the body fluid discharge promoting medicine storage unit 31 and the medicine supplying mechanism 33, where the switching valve 67 is connected with the air introducing unit 35 by the air flow path 36, and the calibration fluid storage unit 38 by the calibration fluid flow path 39. The air introducing unit 35 is a ventilation hole opened to atmosphere. The switching valve 67 can be switched to a state in which the body fluid discharge promoting medicine storage unit 31 side and the medicine supplying mechanism 33 side are communicated and the air introducing unit 35 side and the calibration fluid storage unit 38 side are closed, a state in which the air introducing unit 35 side and the medicine supplying mechanism 33 side are communicated and the body fluid discharge promoting medicine storage unit 31 side and the calibration fluid storage unit 38 side are closed, and a state in which the calibration fluid storage unit 38 side and the medicine supplying mechanism 33 side are communicated and the body fluid discharge promoting medicine storage unit 31 side and the air introducing unit 35 side are closed. The open/close valve 43 is arranged on the medicine flow path 34.

In the body fluid analyzer 66, the supply and discarding of the calibration fluid and the medicine, and the discarding of the body fluid are carried out in the following manner.

When feeding the calibration fluid to the test unit 30, the open/close valve 43 is opened, the switching valve 67 is switched to the state in which the calibration fluid storage unit 38 side and the medicine supplying mechanism 33 side are communicated, and the medicine supplying mechanism 33 is operated. The calibration fluid in the calibration fluid storage unit 38 is then sent out, and the calibration fluid is supplied into the test unit 30 through the medicine injecting hole 27 and the body fluid extracting unit 23.

When discharging the calibration fluid, the open/close valves 43, 53 are opened, the switching valve 67 is switched to the state in which the air introducing unit 35 side and the medicine supplying mechanism 33 side are communicated, and the medicine supplying mechanism 33 is operated. The air is then injected to the medicine injecting hole 27 so that the calibration fluid in the body fluid extracting unit 23 and the test unit 30 is pushed out by the air pressure and discarded to the discarding body fluid storage unit 52.

When supplying the body fluid discharge promoting medicine α to the body fluid extracting unit 23, the open/close valve 43 is opened, the switching valve 67 is switched to the state in which the body fluid discharge promoting medicine storage unit 31 side and the medicine supplying mechanism 33 side are communicated and the medicine supplying mechanism is operated. The body fluid discharge promoting medicine α in the body fluid discharge promoting medicine storage unit 31 is then sent out, and the body fluid discharge promoting medicine α is supplied into the body fluid extracting unit 23 through the medicine injecting hole 27.

When discharging the body fluid discharge promoting medicine α, the open/close valves 43, 48 are opened, the switching valve 67 is switched to the state in which the air introducing unit 35 side and the medicine supplying mechanism 33 side are communicated, and the medicine supplying mechanism 33 is operated. The air is then injected to the medicine injecting hole 27 so that the discarding medicine in the body fluid extracting unit 23 is pushed out by the air pressure and discarded to the discarding medicine storage unit 47.

When discharging the discarding body fluid in the body fluid extracting unit 23 and the test unit 30, the open/close valves 43, 53 are opened, the switching valve 67 is switched to the state in which the air introducing unit 35 side and the medicine supplying mechanism 33 side are communicated, and the medicine supplying mechanism 33 is operated. The air is then injected to the medicine injecting hole 27 so that the body fluid β in the body fluid extracting unit 23 and the test unit 30 is pushed out by the air pressure and discarded to the discarding body fluid storage unit 52.

Therefore, according to the body fluid analyzer 66, the body fluid collecting mechanism 50 and the medicine collecting mechanism 45 can be eliminated, and hence the structure of the body fluid analyzer 66 can be simplified and the cost can be reduced, and furthermore, the body fluid analyzer 66 can be miniaturized.

### Third Embodiment

A body fluid analyzer 71 according to a third embodiment will now be described.

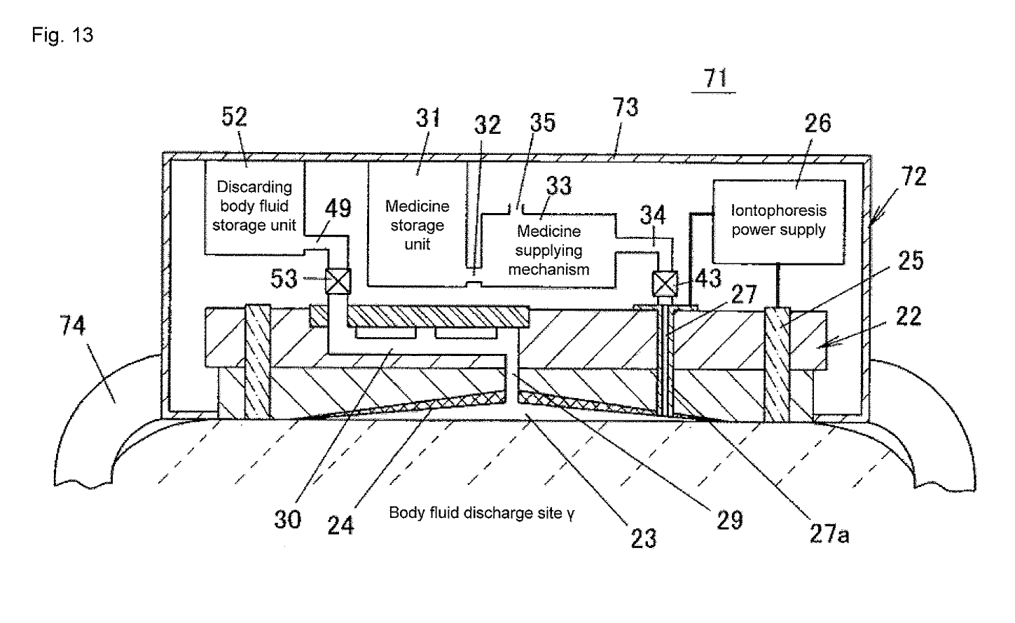

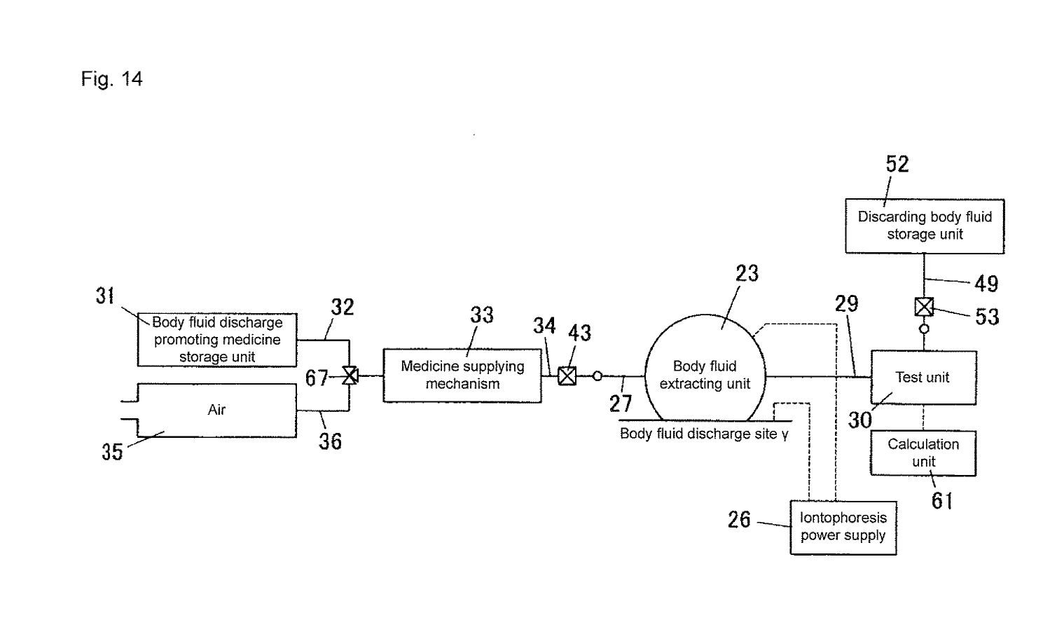

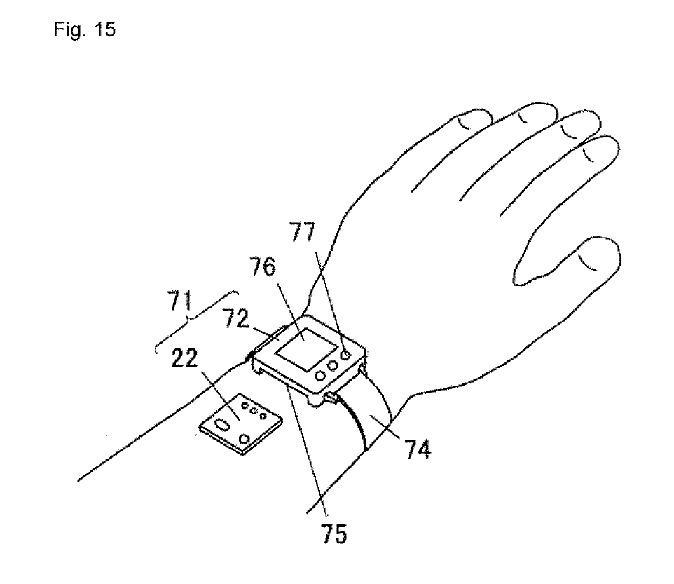

FIG. 13 is a schematic cross-sectional view showing a configuration of a body fluid analyzer 71. FIG. 14 is a view showing a configuration for supplying and discarding the body fluid discharge promoting medicine and discarding the body fluid in the body fluid analyzer 71. FIG. 15 is a perspective view showing a state in which the body fluid analyzer 71 is attached to the arm of the subject.

With reference to FIG. 13, the body fluid analyzer 71 according to the third embodiment discards the discarding medicine to the discarding body fluid storage unit 52 (or collects to the body fluid discharge promoting medicine storage unit 31) in the body fluid analyzer 66 according to the second embodiment, so that the medicine collecting hole 28, the discarding medicine flow path 44, and the discarding medicine storage unit 47 are further unnecessary.

In FIG. 13 and FIG. 14, the illustration of the calibration fluid storage unit 38 is omitted, but the calibration fluid storage unit 38 may be connected to the switching valve 67 in a switchable manner same as the body fluid analyzer 66 according to the second embodiment if the calibration fluid needs to be used, and the calibration fluid may be supplied and collected similar to the body fluid analyzer 66.

In the body fluid analyzer 71, the body fluid discharge promoting medicine α in the body fluid discharge promoting medicine storage unit 31 can be supplied to the body fluid extracting unit 23, similar to the body fluid analyzer 66.

In other words, when discharging the body fluid discharge promoting medicine α remaining in the body fluid extracting unit 23, the open/close valves 43, 53 are opened, the switching valve 67 is switched to the state in which the air introducing unit 35 side and the medicine supplying mechanism 33 side are communicated, and the medicine supplying mechanism 33 is operated. The air is then injected to the medicine injecting hole 27, the discarding medicine in the body fluid extracting unit 23 is pushed out by the air pressure, passed through the test unit 30, and discarded to the discarding body fluid storage unit 52.

In another method, the open/close valve 43 may be opened, and the body fluid discharge promoting medicine α in the body fluid extracting unit 23 may be aspirated with the medicine supplying mechanism 33 so that the used body fluid discharge promoting medicine α is collected to the body fluid discharge promoting medicine storage unit 31. According to this method, the consuming amount of the body fluid discharge promoting medicine α can be reduced since the body fluid discharge promoting medicine α can be reused.

When discharging the discarding body fluid in the body fluid extracting unit 23 and the test unit 30, the body fluid β in the body fluid extracting unit 23 and the test unit 30 is pushed out with the air pressure and discarded to the discarding body fluid storage unit 52, similar to the body fluid analyzer 66.

Therefore, according to the body fluid analyzer 71 the medicine collecting hole 28 and the discarding medicine storage unit 47 can be further eliminated, and hence the structure of the body fluid analyzer 66 can be further simplified and the cost can be reduced, and furthermore, the body fluid analyzer 66 can be further miniaturized.

In the body fluid analyzer 71, the tube 27a made from a conductive material such as metal is embedded along the up and down direction in the body fluid collecting chip 22 where the medicine injecting hole 27 is formed by the tube 27a, and the lower end of the tube 27a is electrically contacting the administration electrode 24. One output terminal of the iontophoresis power supply 26 is connected to the tube 27a. Thus, in the body fluid analyzer 71, the voltage can be applied between the administration electrodes 24, 25 through the tube 27a.

The body fluid analyzer 71 is divided to the body fluid collecting chip 22 (i.e., body fluid collecting unit) including the body fluid extracting unit 23, the administration electrode 24, the administration electrode 25, the medicine injecting hole 27, the body fluid feeding path 29 and the test unit 30, and the analyzer main body 72 including the body fluid discharge promoting medicine storage unit 31, the medicine supplying mechanism 33, the discarding body fluid storage unit 52, the open/close valves 43, 53, the iontophoresis power supply 26, and the battery for driving the medicine supplying mechanism 33.

As shown in FIG. 13 and FIG. 15, the analyzer main body 72 is accommodated in the casing 73 and can be attached to the arm of the subject by the band 74. Furthermore, the analyzer main body 72 includes a display unit 76 for displaying test results, and the like, and an operation button 77 for inputting test start or switching the display.

The body fluid collecting chip 22 can be attached or separated to the interior of the analyzer main body 72 from the chip inserting portion 75. The lower surface of the body fluid collecting chip 22 is closely attached to the body fluid discharge site γ as shown in FIG. 13 when the body fluid collecting chip 22 is attached to the analyzer main body 72. When the body fluid collecting chip 22 is attached inside the analyzer main body 72, the output terminal of the iontophoresis power supply 26 is brought into contact with the upper surface of the tube 27a and the administration electrode 25, the medicine flow path 34 for supplying medicine is connected to the medicine injecting hole 27, and the discarding body fluid flow path 49 for collecting the body fluid is connected to the body fluid feeding path 29.

The body fluid discharge promoting medicine storage unit 31 and the discarding body fluid storage unit 52 may be arranged on the body fluid collecting chip 22. The test unit 30 may be arranged in the analyzer main body 72.

According to such configuration, the test can be continuously carried out over a plurality of times with the analyzer main body 72 attached to the arm or the like of the subject. For instance, the body fluid can be repeatedly collected without changing the body fluid collecting chip 22 if it is only the collecting of the body fluid, and the test can be continuously carried out over a plurality of times without changing the body fluid collecting chip 22 even when carrying out the test as long as the function of the enzyme lasts. Furthermore, the test can be continued by simply changing the body fluid collecting chip 22 or the test unit cover 22c even when the action of the enzyme becomes weak.

### Variant of Third Embodiment

First a few variants of the administration electrode 24 will be shown.

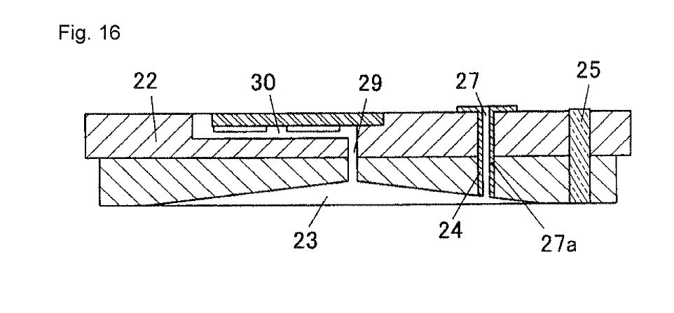

In the variant shown in FIG. 16, the conductive film arranged on the inner wall surface of the body fluid extracting unit 23 is removed and the tube 27a for forming the medicine injecting hole 27 is formed by a conductive material. The output terminal of the iontophoresis power supply 26 is connected to the tube 27a so that the tube 27a can be used as the administration electrode 24. The voltage can be applied to the body fluid discharge promoting medicine in the body fluid extracting unit 23 by the tube 27a or the administration electrode 24 since the lower end of the tube 27a is reached to the inner wall surface of the body fluid extracting unit 23.

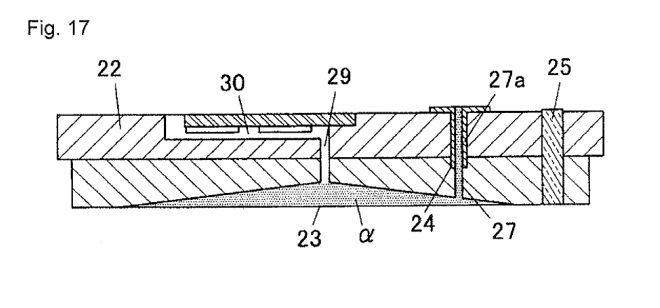

As shown in FIG. 17, the tube 27a made of conductive material may not have the lower end reached to the body fluid extracting unit 23. This is because the voltage can be applied to the body fluid discharge promoting medicine α in the body fluid extracting unit 23 through the body fluid discharge promoting medicine α remaining in the medicine injecting hole 27 even if the tube 27a is short.

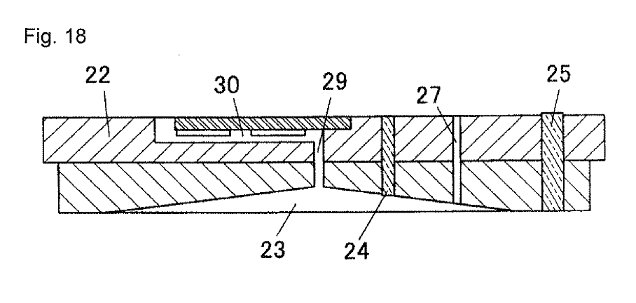

Moreover, as in the variant shown in FIG. 18, the conductive film arranged on the inner wall surface of the body fluid extracting unit 23 may be removed and the administration electrode 24 may be arranged at a position different from the medicine injecting hole 27.

Obviously, with the electrode structure shown in FIGS. 16 to 18, if air bubbles form at one part of the body fluid extracting unit 23 or the medicine injecting hole 27, the administration electrode 24 and the body fluid discharge promoting medicine α are electrically separated by the air bubbles and voltage may not be applied to the body fluid discharge promoting medicine. Therefore, the administration electrode 24 desirably has a large contacting area of a certain extent with the body fluid discharge promoting medicine as with the administration electrode arranged in the body fluid analyzer 71 according to the third embodiment (FIG. 13).

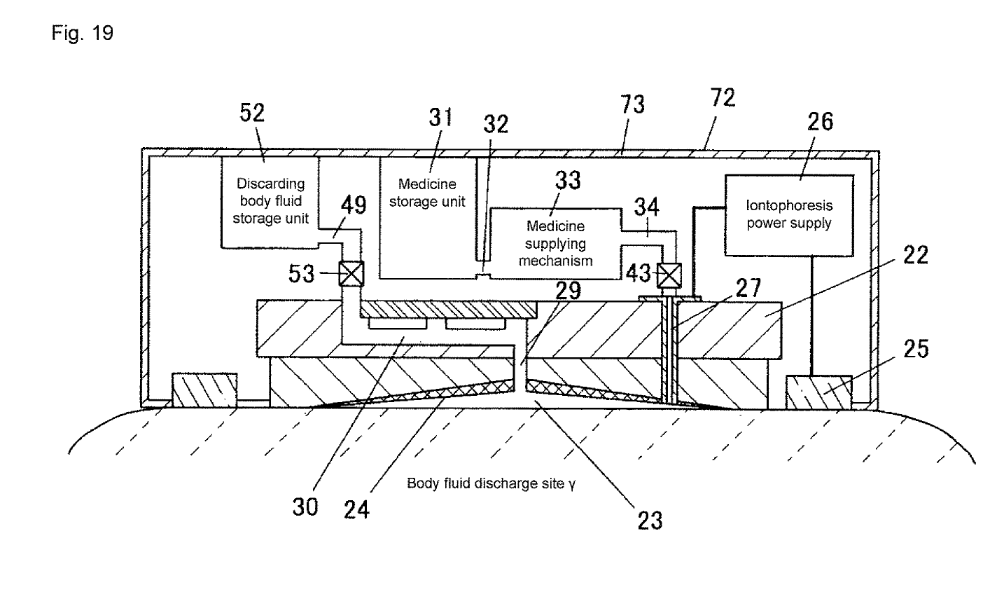

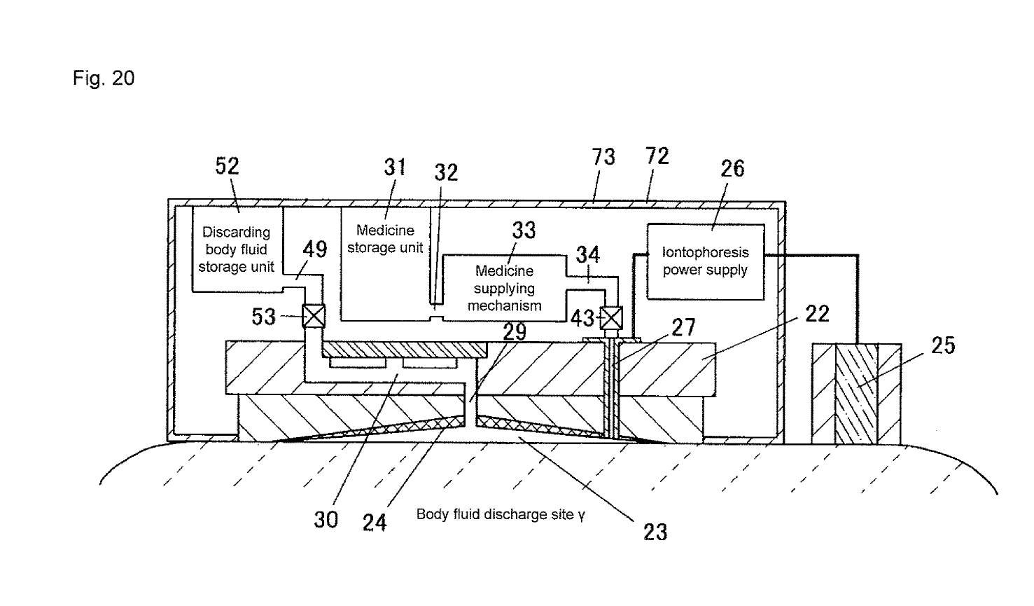

As shown in the variant of FIG. 19, the other administration electrode 25 may be arranged in the analyzer main body 72 instead of the body fluid collecting chip 22. The administration electrode 25 may be arranged to contact the subject at the exterior of the analyzer main body 72, as shown in FIG. 20.

The electrode structure shown in FIGS. 16 to 20 may be applied to the body fluid analyzer of other embodiments.

In another further variant of the body fluid analyzer 71 according to the third embodiment, the analyzer main body 72 and the body fluid collecting chip 22 are not separated, and all configurations such as the body fluid extracting unit 23, the medicine supplying mechanism 33, the test unit 30, and the discarding body fluid storage unit 52 may be arranged on the body fluid collecting chip 22 for attaching to the body fluid discharge site to integrate the body fluid analyzer.

### Fourth Embodiment

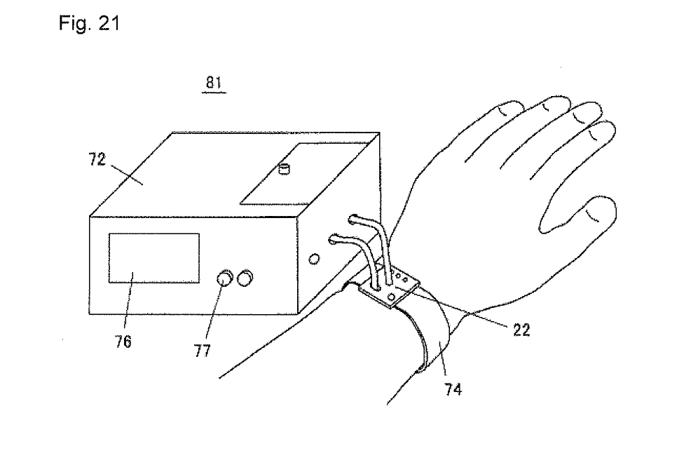

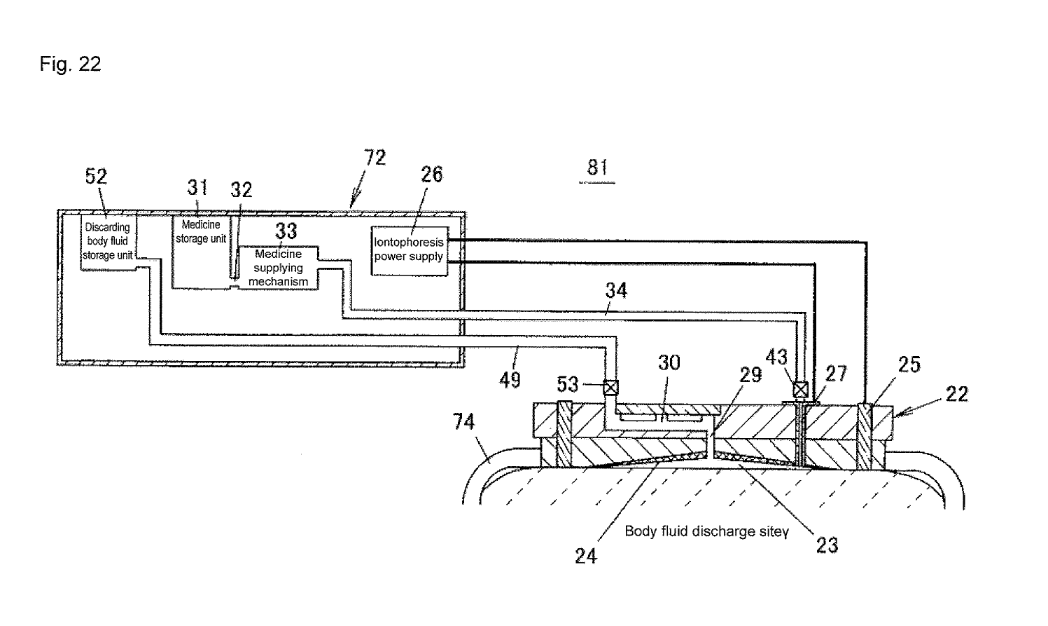

FIG. 21 is a perspective view showing a body fluid analyzer 81 according to a fourth embodiment. FIG. 22 is a schematic cross-sectional view showing a structure of the body fluid analyzer 81.

With reference to FIG. 22, the body fluid analyzer 81 according to the fourth embodiment has the body fluid collecting unit formed in the body fluid collecting chip 22 and the analyzer main body 72 separately configured. As shown in FIG. 21, the body fluid collecting chip 22 includes a band 74, so that the body fluid collecting chip 22 can be attached to the arm or the like of the subject. As shown in FIG. 21, the analyzer main body 72 is amounting type.

According to such configuration, the liquid collecting unit to be attached to the subject can be miniaturized and made lighter.

### Fifth Embodiment

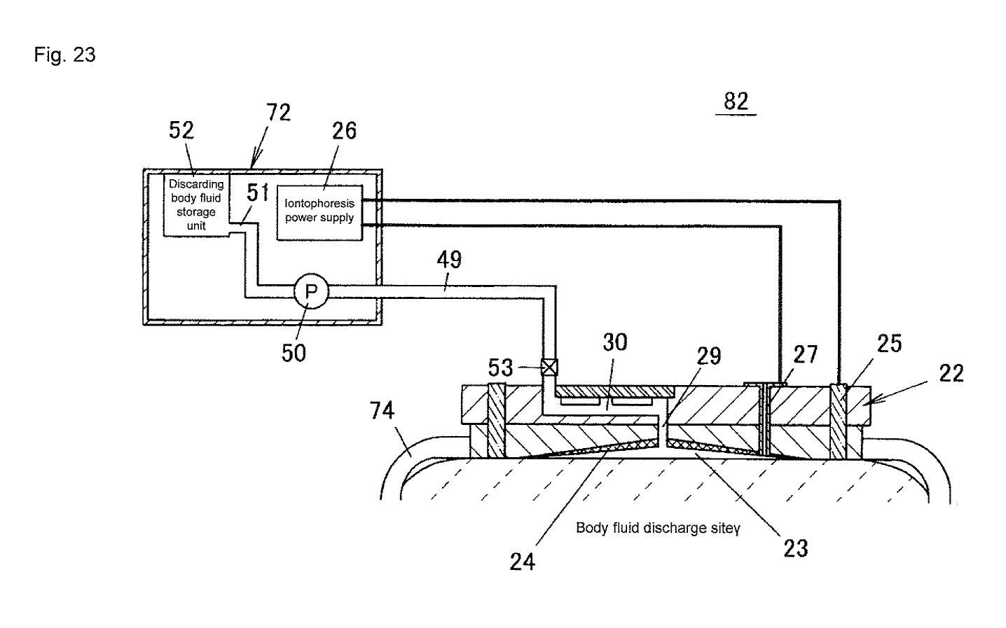

FIG. 23 is a schematic cross-sectional view showing a structure of a body fluid analyzer 82 according to a fifth embodiment. In the body fluid analyzer 82, when supplying the calibration fluid to the test unit 30 or when supplying the body fluid discharge promoting medicine to the body fluid extracting unit 23, they are manually injected from the medicine injecting hole 27 using the syringe or the injector.

When collecting the calibration fluid, the body fluid discharge promoting medicine, and the body fluid, they are aspirated using the body fluid collecting mechanism 50 arranged in the discarding body fluid flow path 49 and discarded to the discarding body fluid storage unit 52.

According to such configuration, the structure of the body fluid analyzer 82 can be simplified and the cost of the body fluid analyzer 82 can be lowered.

The embodiments disclosed herein are illustrative in all aspects and should not be considered restrictive. The scope of the present invention is defined by the Claims rather than by the description made above, and all modifications of the meaning equivalent to the Claims and within the scope of the Claims are intended to be encompassed herein.

### INDUSTRIAL APPLICABILITY

The present invention is suitably used as a body fluid collecting device for collecting body fluid such as sweat. The present invention is also suitably used as a body fluid analyzer for analyzing a specific component contained in the body fluid. Furthermore, the present invention can be used as a medical equipment for diagnosing illnesses such as diabetes.

### DESCRIPTION OF SYMBOLS

11 holding member
12 bio sensor chip
13 substrate
14a, 14b comb tooth-like electrode
15 protective electrode
16 enzyme film
17 separation film
18 skin
19 sweat
21 body fluid analyzer
22 body fluid collecting chip
22a upper plate
22b lower plate
22c test unit cover
23 body fluid extracting unit
24, 25 administration electrode
26 iontophoresis power supply
27 medicine injecting hole
27a tube
28 medicine collecting hole
29 body fluid feeding path
30 test unit
31 body fluid discharge promoting medicine storage unit
32 medicine flow path
33 medicine supplying mechanism
34 medicine flow path
35 air introducing unit
36 air flow path
38 calibration fluid storage unit
39 calibration fluid flow path
40 calibration fluid supplying mechanism
41 calibration fluid flow path
42 switching valve
43 open/close valve
44 discarding medicine flow path
45 medicine collecting mechanism
46 discarding medicine flow path
47 discarding medicine storage unit
48 open/close valve
49 discarding body fluid flow path
50 body fluid collecting mechanism
51 discarding body fluid flow path
52 discarding body fluid storage unit
53 open/close valve
54, 55 test electrode
56 ammeter
57 enzyme
58 color forming dye
59 light projecting unit
60 light receiving unit
61 calculation unit
66 body fluid analyzer
67 switching valve
71 body fluid analyzer
72 analyzer main body
73 casing
74 band
75 chip inserting portion
76 display unit
77 operation button
81 body fluid analyzer
82 body fluid analyzer

1. A body fluid collecting device for extracting and collecting body fluid from under the skin or from the body of a subject, the body fluid collecting device comprising:
a body fluid extracting unit having a function of holding a body fluid discharge promoting medicine for discharging the body fluid from under the skin or from the body on a body fluid discharge site and a function of collecting the body fluid extracted from a site where the body fluid discharge promoting medicine is administered;
a medicine collecting passage, having one end opened at the body fluid extracting unit, for collecting or discarding the body fluid discharge promoting medicine; and
a medicine collecting mechanism for collecting or discarding the body fluid discharge promoting medicine remaining in the body fluid extracting unit after administered to under the skin or into the body through the medicine collecting passage.

2. The body fluid collecting device according to claim 1, further comprising a medicine injection passage for injecting the body fluid discharge promoting medicine to the body fluid extracting unit.

3. The body fluid collecting device according to claim 2, further comprising:
a body fluid discharge promoting medicine storage unit for storing the body fluid discharge promoting medicine; and
a medicine supplying mechanism for supplying the body fluid discharge promoting medicine stored in the body fluid discharge promoting medicine storage unit from the medicine injection passage to the body fluid extracting unit.

4. The body fluid collecting device according to claim 3, wherein
the body fluid discharge promoting medicine is liquid; and
the medicine supplying mechanism is a pump.

5. The body fluid collecting device according to claim 3, wherein
the body fluid discharge promoting medicine is liquid;
the body fluid discharge promoting medicine storage unit is a breakable liquid container in which the body fluid discharge promoting medicine is enclosed; and
the medicine supplying mechanism is a breaking tool for breaking the liquid container.

6. The body fluid collecting device according to claim 2, wherein a valve for opening and closing the passage is arranged on the medicine injection passage.

7. The body fluid collecting device according to claim 1, wherein
the body fluid discharge promoting medicine is liquid; and
the medicine collecting mechanism collects or discards the body fluid discharge promoting medicine from the body fluid extracting unit by a method selected from a method of pushing out the body fluid discharge promoting medicine by air sent to the body fluid extracting unit using a pump, a method of pushing out the body fluid discharge promoting medicine by the body fluid discharged from under the skin or from the body to the body fluid extracting unit, and a method of aspirating the body fluid discharge promoting medicine using a pump.

8. The body fluid collecting device according to claim 1, wherein
the body fluid discharge promoting medicine is liquid; and
the medicine collecting mechanism feeds a volatile liquid to the body fluid extracting unit to substitute or mix with the body fluid discharge promoting medicine, and discards the body fluid discharge promoting medicine from the body fluid extracting unit by volatilizing the volatile liquid.

9. The body fluid collecting device according to claim 1, further comprising:
a body fluid collecting passage, having one end opened at the body fluid extracting unit, for collecting the body fluid; and
a body fluid collecting mechanism for collecting the body fluid collected in the body fluid extracting unit through the body fluid collecting passage.

10. The body fluid collecting device according to claim 9, wherein an inner wall surface of the body fluid extracting unit is inclined so that an open end of the body fluid collecting passage is positioned at the top of the body fluid extracting unit at a cross-section of the body fluid extracting unit including a direction perpendicular to the body fluid discharge site.

11. The body fluid collecting device according to claim 9, wherein a valve for opening and closing the passage is arranged on the body fluid collecting passage.

12. The body fluid collecting device according to claim 1, further comprising at least two administration electrodes for flowing current between the body fluid discharge promoting medicine held in the body fluid extracting unit and the body fluid discharge site.

13. The body fluid collecting device according to claim 12, wherein at least a portion close to the body fluid extracting unit of the medicine injection passage for injecting the body fluid discharge promoting medicine to the body fluid extracting unit is formed by a conductive material so that the medicine injection passage also serves as either one of the administration electrodes.

14. The body fluid collecting device according to claim 12, further comprising:
a medicine injection passage for injecting the body fluid discharge promoting medicine to the body fluid extracting unit; wherein
at least one of the administration electrodes is arranged to contact the body fluid discharge promoting medicine held in the body fluid extracting unit and at a position different from the medicine injection passage.

15. The body fluid collecting device according to claim 12, wherein a conductive film is arranged on the surface of the body fluid extracting unit, and at least one of the administration electrodes is formed by the conductive film.

16. The body fluid collecting device according to claim 1, further comprising a mechanism for generating an ultrasonic vibration between the body fluid discharge promoting medicine held in the body fluid extracting unit and the body fluid discharge site.

17. The body fluid collecting device according to claim 1, wherein
the body fluid discharge promoting medicine is medicine containing pilocarpine or acetylcholine; and
the body fluid extracted and collected from under the skin or from the body is sweat.

18. A body fluid collecting method for collecting body fluid using the body fluid collecting device according to claim 1, the body fluid collecting method comprising the steps of:
attaching the body fluid collecting device to a body fluid discharge site; and sequentially executing with the body fluid collecting device processes of
administering a body fluid discharge promoting medicine held in a body fluid extracting unit to under the skin or into the body;
removing the body fluid discharge promoting medicine remaining in the body fluid extracting unit with a medicine collecting mechanism; and
collecting the body fluid extracted from the site where the body fluid discharge promoting medicine is administered with the body fluid extracting unit.

19. A body fluid analyzer for detecting or measuring a specific component in a body fluid collected from under the skin or from the body of a subject, wherein
the body fluid collecting device according to claim 1 is arranged as a body fluid collecting unit for collecting from under the skin or from the body of the subject; and the body fluid analyzer further comprises:
a test unit for detecting or measuring the specific component in the body fluid collected by the body fluid extracting unit arranged in the body fluid collecting unit.

20. The body fluid analyzer according to claim 19, wherein
the test unit includes an enzyme that specifically reacts with the specific component in the body fluid, and a test electrode; and
a signal based on a current generated from a reaction between the specific component in the body fluid and the enzyme is detected by the test electrode to detect or measure the specific component in the body fluid.

21. The body fluid analyzer according to claim 19, wherein
the test unit includes an enzyme and a color forming dye that specifically react with the specific component in the body fluid; and
a color reaction between the specific component in the body fluid and the enzyme and the color forming dye is optically detected to detect or measure the specific component in the body fluid.

22. The body fluid analyzer according to claim 20, wherein
two or more test units are arranged; and
the test units include enzymes of different type from each other.

23. The body fluid analyzer according to claim 20, wherein
two or more test units are arranged; and
the test units include same type of enzyme.

24. The body fluid analyzer according to claim 20, wherein
four or more test units are arranged; and
with a combination of test units having different types of enzymes from each other as one set, the one set of test units is arranged in plurals.

25. The body fluid analyzer according to claim 19, further comprising:
a calibration fluid storage unit for storing calibration fluid for calibrating the test unit; and
a calibration fluid supplying mechanism for supplying the calibration fluid stored in the calibration fluid storage unit to the test unit.

26. The body fluid analyzer according to claim 19, further comprising:
a body fluid collecting passage, having one end opened at the body fluid extracting unit, for collecting the body fluid;
a discarding body fluid storage unit for discarding the body fluid after the test; and
a body fluid collecting mechanism for collecting the body fluid collected at the body fluid extracting unit through the body fluid collecting passage, and discarding the body fluid after the test by the test unit to the discarding body fluid storage unit.

27. The body fluid analyzer according to claim 19, further comprising a test unit having glucose oxidase or glucose dehydrogenating enzyme as enzyme.

28. The body fluid analyzer according to claim 19, further comprising a test unit having glucose oxidase or glucose dehydrogenating enzyme as enzyme, and a test unit having lysine oxidase as enzyme.

29. The body fluid analyzer according to claim 19, further comprising:
a medicine supplying mechanism for supplying the body fluid discharge promoting medicine stored in the body fluid discharge promoting medicine storage unit to the body fluid extracting unit;
a discarding body fluid storage unit for discarding the body fluid after the test; and
a body fluid collecting mechanism for collecting the body fluid collected in the body fluid extracting unit, and discarding the body fluid after the test by the test unit to the discarding body fluid storage unit.

30. The body fluid analyzer according to claim 29, wherein the body fluid collecting passage also serves as the medicine collecting passage, the discarding body fluid storage unit also serves as the discarding medicine storage unit, and the medicine collecting mechanism uses the medicine supplying mechanism.

31. The body fluid analyzer according to claim 29, wherein the body fluid collecting mechanism uses the medicine supplying mechanism.

32. The body fluid analyzer according to claim 29, further comprising:
a body fluid collecting chip for attaching to a body fluid discharge site; and
an analyzer main body to be stationed; wherein
the body fluid collecting chip includes the body fluid extracting unit and the test unit; and
the analyzer main body includes the medicine supplying mechanism, the medicine collecting mechanism, the body fluid collecting mechanism, and the discarding body fluid storage unit.

33. The body fluid analyzer according to claim 29, further comprising:
an analyze main body for attaching to a body fluid discharge site; and
a body fluid collecting chip removably attached to the analyzer main body; wherein
the body fluid collecting chip includes the body fluid extracting unit and the test unit; and
the analyzer main body includes the medicine supplying mechanism, the medicine collecting mechanism, the body fluid collecting mechanism, and the discarding body fluid storage unit.

34. The body fluid analyzer according to claim 29, wherein the body fluid extracting unit, the medicine supplying mechanism, the medicine collecting mechanism, the test unit, the body fluid collecting mechanism, and the discarding body fluid storage unit are arranged on the body fluid collecting chip for attaching to the body fluid discharge site.
